(19) United States
(12) Patent Application Publication
(10) Pub. No.: US 2016/0263263 A1
(43) Pub. Date: Sep. 15, 2016

(54) Sanitizer

(71) Applicant: Michael E. Robert, Farmington Hills, MI (US)

(72) Inventor: Michael E. Robert, Farmington Hills, MI (US)

(21) Appl. No.: 15/042,770

(22) Filed: Feb. 12, 2016

Related U.S. Application Data
(63) Continuation-in-part of application No. PCT/US15/20288, filed on Mar. 12, 2015.
(60) Provisional application No. 61/952,007, filed on Mar. 12, 2014.
(60) Provisional application No. 61/970,661, filed on Mar. 26, 2014.
(60) Provisional application No. 62/115,373, filed on Feb. 12, 2015.

(51) Int. Cl. A61L 2/14; H01J 37/08; H01J 37/32; A61L 2/26

(52) U.S. Cl. CPC A61L 2/14; A61L 2/26; H01J 37/08; H01J 37/32

(57) ABSTRACT

A sanitizer for sanitizing various surfaces including hands, hardware, fixtures, appliances, countertops, equipment, utensils and more and more specifically to a chemical-free sanitizer, more specifically to an ozone-free sanitizer and yet more specifically to an electronic sanitizer and yet more specifically to an ion source sanitizer.

### CROSS REFERENCE TO RELATED APPLICATIONS

This Continuation-In-Part patent application claims the benefit of and priority to PCT Patent Application International Serial No. PCT/US 15/20288 filed Mar. 12, 2015 entitled “Sanitizer,” which claims the benefit of and priority to U.S. Provisional Patent Application Ser. No. 61/952,007 filed Mar. 12, 2014 entitled “Sanitizer,” U.S. Provisional Patent Application Ser. No. 61/970,661 filed Mar. 26, 2014 entitled “Ion Generator,” and U.S. Provisional Patent Application Ser. No. 62/115,373 filed Feb. 12, 2015 entitled “Ion Generator,” and U.S. Provisional Patent Application Ser. No. 62/181,475 filed Jun. 18, 2015 entitled “Sanitizer With an Ion Generator and Ion Electrode Assembly,” the entire disclosures of which are incorporated herein by reference.

### BACKGROUND OF THE INVENTION

1. Field of the Invention

The present invention is directed to a sanitizer for sanitizing various surfaces including hands, hardware, fixtures, appliances including interiors of refrigerators, countertops, equipment, utensils and specifically to a chemical-free sanitizer, more specifically to an ozone-free sanitizer and yet more specifically to an electronic sanitizer and yet more specifically to an ion source sanitizer, and even more specifically, an ion sanitizer that does not include fans or other mechanisms of air propulsion, and does not use sacrificial anodes and cathodes.

2. Description of the Prior Art

It is well known that many infectious diseases and pathogens are communicated through touch or contact. Therefore, commonly touched items in public areas and facilities such as doorknobs, handles, fixtures, and other surfaces may spread such infectious diseases and pathogens. People are particularly concerned with touching various surfaces in public restrooms even communal restrooms at a work place due to actual or perceived sanitary conditions of those restrooms and the users of the restrooms. However, contact with door handles, knobs and other fixtures related to the restroom is many times unavoidable. Other exemplary surfaces that may be unavoidable and be contaminated with pathogens from people or other sources including food preparation may include drinking fountains, kitchen counter tops, shared appliances, refrigerator shelves, and nearly any other surface that multiple people may contact. Therefore, many people generally find it desirable to avoid or minimize contact with such surfaces when possible.

People are particularly concerned with the cleanliness of surfaces after washing their hands or before the eating of food. However, touching many of the surfaces in a restroom after washing hands or in a kitchen while preparing food particularly in a work place kitchen is unavoidable. For example, in most restrooms as a person must touch the handle of the door to exit a restroom, touch the same faucet handle used to turn on the water or to turn off the faucet which may recontaminate the just cleaned hands. In a kitchen, other than door and fixture handles such as faucets, a refrigerator door handle or the surface of a microwave and light switches may all be contaminated with various pathogens. Some people use extra paper towels to cover and touch handles of door or faucets in certain situations, however, generally this is wasteful and adds expense for the facility including increased paper cost as well as increased labor cost for replacing the paper products more frequently.

A number of prior methods have been proposed, all having limited success or significant drawbacks in sanitizing various surfaces including door handles. The first method is generally more frequent cleaning of such surfaces, however, this increases labor costs and generally people are distrustful that the surfaces have been properly cleaned. In addition, even if the cleaning was thorough and no pathogens exist on the surface, the very first contact by a person may place undesirable infectious agents or pathogens on the surface and any subsequent users may come in contact with such infectious agents or pathogens. Therefore, the more frequent cleanings do not solve the problem of contaminated surfaces.

Some facilities provide various cleaning wipes, liquids, or sponges that may be used for cleaning of the surface by a user. While these are generally capable of cleaning the surface, the use is limited to a person actually using them. A big disadvantage to these wipes, liquids or sponges is that they require frequent replacement thereby increasing the cost for the facility. Many times these anti-bacterial sprays, liquids or wipes are empty creating an undesirable situation for the person using the facility.

To address the above problems, some manufacturers have introduced various electronic chemical sanitizers that with little to no interaction with a user at regular intervals or upon activation of a sensor, sprays a liquid on the desired surface. In addition to the increased maintenance cost as well as product cost of replacing the battery and the chemical or wet material, generally most people find it undesirable to touch a moist or damp surface such as a moist or damp door handle, even if the moisture or liquid is a sanitizing chemical. In addition, many people do not like the smell or have various chemical allergies to the chemical being used on the door handle, making it difficult to use that facility. More specifically, such as in an office setting, if one worker has a chemical allergy to the cleaning device that is being used, which on a timed or activated interval sprays a door handle, it may prevent further use in that facility. To address the problems some people have proposed using ultraviolet sanitizers that when positioned or placed over a non-porous surface effectively sterilizes and sanitizes the surface. While such devices prevent the spread of pathogens passed on by contact by direct exposure to ultraviolet light, these devices generally are power intensive and require frequent battery changes or recharging unless they are hardwired into a facility's electrical system. Therefore, for doors, wherein they are controlled by a preprogrammed timer or motion sensing, their useful life is relatively limited requiring regular maintenance by the facility thereby raising costs. Many people are also concerned regarding sticking their hands on a door handle to open it where it will be bathed in ultraviolet light. The positioning of many of these devices is above a door handle or counter top which places it high enough that smaller people, such as children, may inadvertently look directly at the ultraviolet lamp which is undesirable and could cause vision issues. Therefore, the implementation of these devices as sanitizers for various fixtures that cannot fit in an enclosure has been limited due to their serious drawbacks.

To address the shortcomings with various chemical and ultraviolet light sanitizers, some manufacturers have introduced ozone sanitizers, which is known to be a potent sanitizer for various surfaces as it is a highly reactive oxidizer. Ozone works well at killing various pathogens, and unlike chemical sanitizers, leaves no chemical residue on the treated surfaces. Ozone has been highly desirable for use in food processing plants, but has had limited other practical applications. A sanitizing processing system is generally of limited use because it must control the output of ozone in a sealed environment. Therefore, it is used in large industrial only settings and have not been successfully implemented in households or small commercial applications. More specifically, the application of ozone sanitizing systems has been extremely limited by the more recent understanding that ozone may cause various health issues including according to the EPA, respiratory issues such as lung function, decrements, inflammation and permeability, susceptibility to infection, cardiac affects and more seriously respiratory symptoms including medication use, asthma attacks and more. The respiratory symptoms can include coughing, throat irritation, pain, burning, or discomfort in the chest when taking a deep breath, chest tightness, wheezing or shortness of breath. For some people, more acute or serious symptomatic responses may occur. As the concentration at which ozone effects are first observed depends mainly on the sensitivity of the individual even some parts per billion exposure may cause noticeable issues. Therefore, other than commercial environments where the ozone application must be specifically controlled, and these systems are not desirable for a broader implementation in homes, work places and other facilities, where the ozone is not easily contained, such as functioning as a door handle sanitizer for an operational door.

Existing sanitizers or ozone devices, especially DC sanitizers require a method of propelling the ions or ozone away from the device. As such, many of these devices use fans, compressed air, or other mechanisms for dispersing the ions. One problem with such systems is that in applications where an external power source is not readily available, batteries for fans and other means of propulsion such as CO₂ canisters must be replaced on a fairly regular basis. In mechanisms using a fan powered by battery, the fans substantially limits the life of the battery to the point where it needs to be replaced weekly or even bi-weekly in certain environments. Other systems using compressed air or CO₂ require replacement or recharging of the cartridges or tanks on a regular basis. In addition, any sanitizer requiring a mechanism for propelling the ions outward such as the battery-powered fans or compressed air stop efficiently functioning, without the mechanism for propulsion.

Bipolar ionizers use a high voltage to create an electric field across two discharge points. One point creates positive ions and the other point creates negative ions. It is well known that as the number of points increase, the amount of ions that may be generated due to the nature of electrical fields and increase in surface area from using multiple points, is reduced. More specifically, the use of a single point requires that all of the electrical fields will pass through that point. As such, the production of ions is maximized by use of a single point. Traditionally, multiple points as ion sources were discouraged to maximize ion production.

The most common techniques of creating the required voltage are either a flyback transformer or a voltage multiplier circuit or a combination of the two. Because the high voltage is DC, two discharge points are required—one for positive and the other for negative. Most implementations of a flyback transformer use feedback from a secondary winding on the transformer to create a resonator that switches the primary side of the transformer on and off. While this circuit is simple and cost effective, it often takes long periods of time for the circuit to stabilize and reach its full output.

Therefore, there is a need for an effective sanitizer that does not include the above identified limitations.

### SUMMARY OF THE INVENTION

The present invention is directed to a sanitizer for sanitizing various surfaces including hands, hardware, fixtures, appliances, countertops, equipment, utensils and more and more specifically to a chemical-free sanitizer, more specifically to an ozone-free sanitizer and yet more specifically to an electronic sanitizer and yet more specifically to an ion source sanitizer.

The present invention relates generally to an ion sanitizer including a controller and at least one ion electrode operationally coupled to the controller and the ion electrode includes a plurality of ion sources spaced 6-51 mm apart. The ion sanitizer defines a fixture cavity having a plurality of ion sources each include a point directed toward the fixture cavity. In one embodiment, the at least one ion electrode include a first ion electrode and a second ion electrode and wherein the controller provides a positive DC output to the first ion electrode, and a negative output to the second ion electrode. The ion sources on said first and second electrode face each other and are each directed to said fixture cavity. The ion sanitizer in an AC embodiment further includes a ground electrode spaced at least 10 mm from the ion electrode, and wherein said ground electrode maintains a ground, while said ion electrode fluctuates between positive and negative charge at 1-100 Hz.

The ion sanitizer further includes a housing and the at least one ion electrode is recessed relative to the surface of the housing. The ion sources include a point and which is 0-4 mm recessed relative to said surface of the housing and does not protrude past the surface. In some instances, the housing or a portion thereof may form the ground electrode. The ion sanitizer may include a flexible substrate including at least one conductive element and wherein said ions sources are in electrical communication with the conductive element and on at least one end a controller. The flexible circuit may extend form the controller, similar to LED light strips. The flexible substrate may be coupled to a metallic base forming the ground electrode. In some instances, the metallic base may be a base of a housing, or a mounting member or may even be a conductive metal tape capable of adhering said flexible substrate to a surface. The ion sanitizer may include a plurality of LEDs coupled to the flexible substrate.

The ion sanitizer may include a conductive element and at least a portion of said ion sources are covered with an electrical insulating material. The flexible substrate may have a first longitudinal edge and an opposing second longitudinal edge and wherein the at least one conductive element includes a first conductive element in electrical communication with the ion sources and a second conductive element proximate to one of said first and second edges and wherein said second conductive element is a ground electrode spaced a minimum of 6 mm from the ion sources.

In addition, the ion sanitizer may further including a housing having an outer extent, formed by at least one of a base and a cover and wherein said housing includes a recess on said outer extent configured to receive said at least one ion electrode. The ion electrode may even emit ions up to a full 360 degrees of said outer extent.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a front perspective view of an exemplary sanitizer mounted on a door for sanitizer fixture, such as the illustrated door handle;

FIG. 2 is an exploded perspective view of the sanitizer with a ground reference electrode;

FIG. 3 is an exploded perspective view of the sanitizer in FIG. 1 including two ion source electrodes;

FIG. 4 is a perspective view of the assembled sanitizer without the cover;

FIG. 5 is a front view of the assembled sanitizer without the cover;

FIG. 6 is a bottom view of the sanitizer with the cover removed;

FIG. 7 is a left view of the sanitizer with the cover removed;

FIG. 8 is a front perspective view of an exemplary sanitizer;

FIG. 9 is a left side view of the sanitizer in FIG. 8;

FIG. 10 is an exploded perspective view of the sanitizer in FIG. 8;

FIG. 11 is a bottom view of the sanitizer in FIG. 8;

FIG. 12 is a cross-sectional view along lines 12-12 in FIG. 11;

FIG. 13 is an exploded perspective view of a sanitizer;

FIG. 14 is a side view of the sanitizer in FIG. 22;

FIG. 15 is a cross-sectional view of the sanitizer taken along lines A-A in FIG. 23;

FIG. 16 is a side view of the sanitizer in FIG. 22 showing hidden lines illustrating the locations of the individual components;

FIG. 17 is a bottom view of the sanitizer in FIG. 22 showing hidden lines illustrating the locations of the individual components;

FIG. 18 is a top view of an ion electrode;

FIG. 19 is an end view of the ion electrode in FIG. 18;

FIG. 20 is a top view of an ion electrode;

FIG. 21 is a side view of the ion electrode in FIG. 20;

FIG. 22 is a schematic diagram of flyback converter circuit used to create high voltage DC;

FIG. 23 is a graph of the output of a flyback convertor with 5V square wave input and a 1.25 KV RMS DC Output;

FIG. 24 is a schematic diagram of a flyback convertor using primary feedback to resonate;

FIG. 25 is a voltage multiplier circuit;

FIG. 26 is a step up transformer for high voltage AC supply;

FIG. 27 is a schematic diagram of the ion generator using two flyback transformers;

FIG. 28 illustrates and exemplary input of P1 and P2 from the flyback transformers and the output on the ion electrode;

FIG. 29 is a schematic diagram of an alternative ion generator using two flyback transformers;

FIG. 30 is a partial schematic diagram of the ion generator shown in FIG. 28;

FIG. 31 is a schematic diagram of a simplified version of the ion generator shown in FIG. 28;

FIG. 32 is a diagram of an emitter strip including LEDs;

FIG. 33 is a diagram of an emitter strip with spaced ground electrodes;

FIG. 34 illustrates an exemplary first drive signal and second drive signal and resulting high voltage AC voltage output;

FIG. 35 is a side view of a new ion electrode assembly;

FIG. 36 is an end view of the new ion electrode assembly in FIG. 35;

FIG. 37 is a sectional view of the new ion electrode assembly taken along lines A-A in FIG. 36;

FIG. 38 is an exploded side perspective view of the ion electrode assembly in FIG. 35;

FIG. 39 is a side perspective view of the ion electrode assembly in FIG. 35;

FIG. 40 is a cross sectional view of the ion electrode assembly in FIG. 35;

FIG. 41 is an exploded perspective view of an ion electrode assembly;

FIG. 42 is a perspective view of the assembled ion electrode assembly in FIG. 41;

FIG. 43 is an end view of the ion electrode assembly in FIG. 41;

FIG. 44 is a side view with hidden lines showing the interior components of the ion electrode assembly;

FIG. 45 is a cross sectional view of the ion electrode assembly taken along lines A-A in FIG. 43;

FIG. 46 illustrates the amount of ions produced by the new ion electrode assembly and new ion generator as well as the synthetic wave output in the ion generator; and

FIG. 47 illustrates the amount of ions produced by the new ion electrode assembly and new ion generator as well as the synthetic wave output in the ion generator.

### DETAILED DESCRIPTION

The present invention is generally directed to a sanitizer. The sanitizer generally produces charged ions that are expelled by the sanitizer toward an object or surface to be sanitized using the electrical field of the sanitizer, or in the illustrated DC sanitizer, drawn across the surface and/or fixture to be sanitized. The sanitizer is specifically configured to avoid the production of ozone and should not be confused with ozone sanitizers which sanitize with ozone. Instead, the present invention provides a compact ion sanitizer that avoids the production of ozone during normal operation and therefore sanitizes without any ozone. Careful configuration of the ion sources and voltage is required to avoid the production of ozone during normal operation and as such, the sanitizer does not sanitize with ozone.

Bipolar ionization of a gas creates plasma that is not in thermodynamic equilibrium because the ion temperature is lower than the electron temperature. This plasma is commonly referred to as ‘cold plasma’ or ‘non-thermal plasma’ because it occurs at room temperatures. Plasmas in thermodynamic equilibrium require much more energy and occur at significantly higher temperatures. Cold plasma has many benefits that will be discussed in greater detail. These benefits include, but are not limited to the ability to kill harmful pathogens including bacteria, mycoplasma, viruses, and mold. Additionally, cold plasma may help with a reduction of Volatile Organic Compounds (VOC's) and a reduction of particulates in the air including known allergens. Furthermore, cold plasma also reduces or eliminates static electricity in the air.

Many commercial buildings strive to achieve certification in Leadership in Energy & Environmental Design (LEED). In the process of obtaining LEED certification, improved air quality and low energy usage may assist a building owner in being certified at the highest level possible. Cold plasma is an energy efficient method that may be used to improve air quality. Therefore, it may be possible for a building to achieve a higher LEED certification, for example, when cold plasma is used in connection with heating, ventilation, and air conditioning (HVAC) applications, or other types of sanitizing.

An ion is a molecule that is either positively or negatively charged. Most ions are unstable. A negative ion has at least one extra electron to give up in order to become stable. A positive ion is missing at least one electron that it must gain to become stable. It is believed that such instability of ions creates the desired electrochemistry capable of killing harmful pathogens including, but not limited to bacteria, mycoplasma, viruses, and mold.

Ions created in the air are referred to as ‘air ions’ or sometimes, simply ‘ions’. French physicist Charles Augustin de Coulomb published his paper in 1875 describing the interaction of electrically charged particles. In his research, Coulomb found that a well-insulated conductor, exposed to air soon lost its charge. He concluded that air must be slightly conductive. In 1899 Elster & Geitel discovered the natural existence of ions in the atmosphere. These air ions make the air slightly conductive.

Air ions may be classified by their charge and mobility. An air ion will move in the presence of an electric field due to its charge. The velocity of the air ion is proportional to the strength and direction of the electric field given in Volts per meter (V/m). With velocity given in m/s:

Mobility, μ=(m/s)/(V/m)=m2/Vs

Where; m=distance in meters, s=time in seconds, and V=electrical potential in Volts.

The drift velocity (Vd) of an air ion is proportional to the Electric Field and inversely proportional to its mass. Therefore, smaller ions in a large electric field will have the greatest drift velocity.

Examples of air ions include small stable negative ions such as an Oxide molecule ion (O2−+(H2O)n), Carbon dioxide ion (CO3−+(H2O)n), and Nitric acid ion (NO3−+(H2O)n). Other examples of air ions include small stable positive ions such as a Hydrogen ion (H++(H2O)n), and Oxonium ion (H3O++(H2O)n). Additional examples of air ions include radicals such as Hydroxyl Radical (OH.).

Naturally occurring negative ions may also come from evaporating water and natural events such as lighting, rainstorms and high winds. Air ions may also be artificially created. As in nature, ionization occurs by adding energy to a gas. Examples of different technology used to create ionization are described below.

Electrostatic Precipitator technology uses an electrostatic precipitator to create charged particles that attach to airborne pollutants, but, unlike an ionizer, it captures the contaminants on collector plates instead of surrounding surfaces. Regular cleaning of its collector plates are necessary to keep it operating efficiently.

Photocatalytic Oxidation (PCO) is a technology used to chemically manufacture positive and negative ions using UV radiation shined onto either Titanium Dioxide (TiO2) or a combination of TiO2 and other metals to create a catalytic reaction. This chemical reaction creates negative and positive ions.

Dielectric Barrier Discharge technologies also known as silent discharge or ozone discharge is the electrical discharge between two electrodes separated by an insulating dielectric barrier that creates ionization. Ernst Werner von Siemens discovered it in 1857. In the common coaxial configuration, the dielectric is shaped in the same form as common fluorescent tubing. It is filled at atmospheric pressure with either a rare gas or rare gas-halide mix, with the glass walls acting as the dielectric barrier. Due to the atmospheric pressure level, such processes require high energy levels to sustain. The glass tubes are fragile, expensive and need regular replacement.

Needle Point technology is the most simple, cost effective and energy efficient method of bipolar ionization. A high voltage AC or DC source is applied to needles. High voltages applied to a non-grounded conductive surface will build up a positive or negative change on that surface. If the surface has a sharp tip with near-zero surface area there will not be enough surface to hold the charge and the energy of the charge will be dissipated into the surrounding air to create ions.

The sanitizer 30 generally includes a housing having a cover 50 and a backplate or base 40. The housing is generally meant to protect the interior components and provide a pleasing look and feel to the sanitizer 30. Of course, the housing may be made in any size, shape, style, or configuration, such as to blend in with the surrounding style or décor. In some embodiments where the sanitizer 30 itself is hidden or protected, such as under a shelf, inside appliances, under a cabinet and the like, the sanitizer 30 may be formed without a housing. The base 40 of the housing may also be configured in any size, shape or configuration and may be formed to fit to or attach to a variety of surfaces 10 including contoured surfaces. The base 40 is generally used to mount the sanitizer 30 to another surface 10 such as a door 12, wall, fixture 20 or proximate to any other surface 10 or fixture 20 requiring sanitization. Of course, it is possible to mount the sanitizer 30 out of sight yet proximate to the surface 10 to be sanitized without requiring certain portions of the housing.

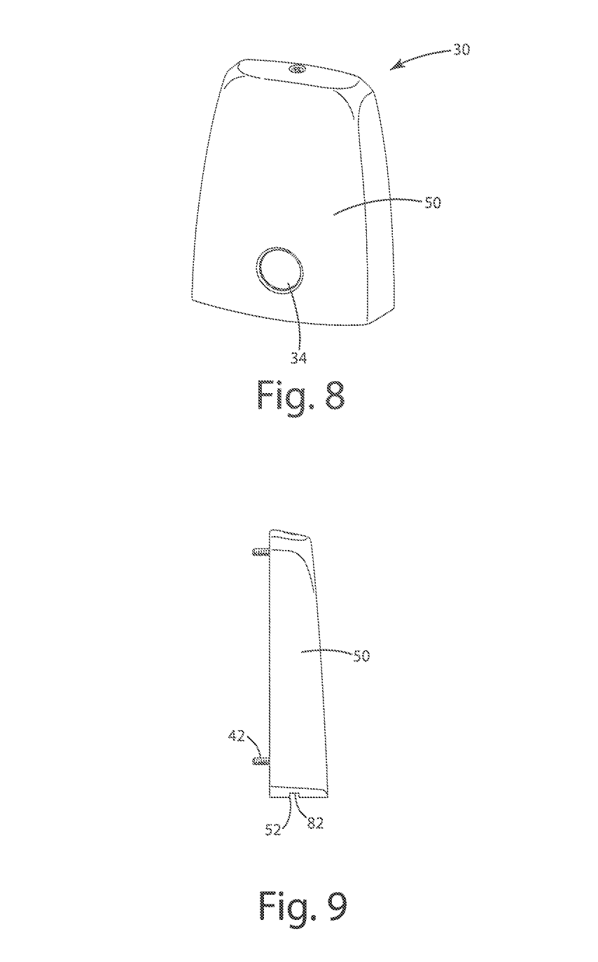

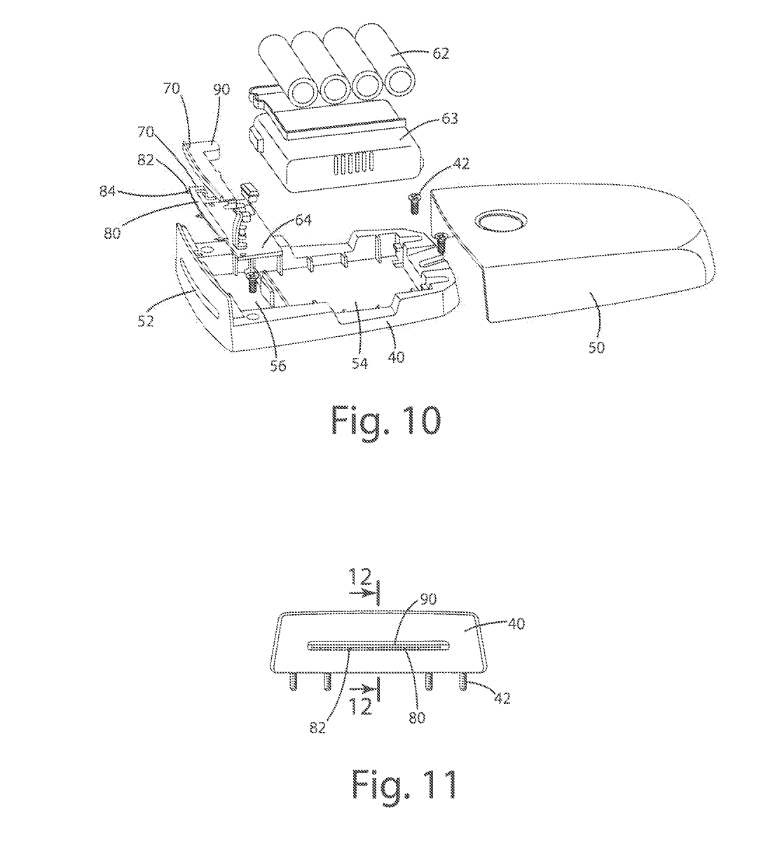

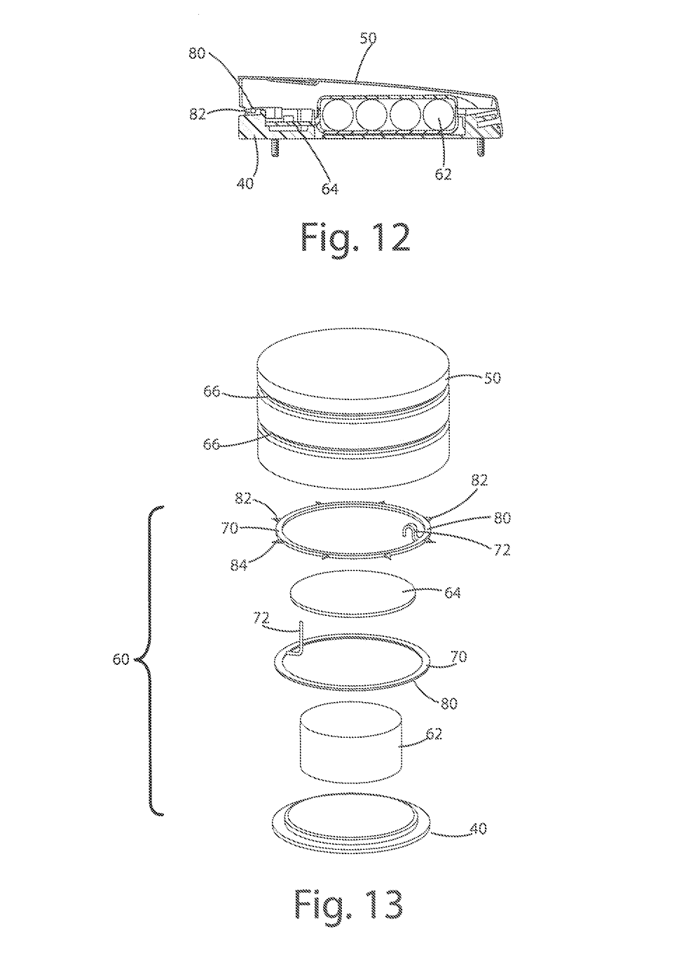

As illustrated in FIG. 8, the sanitizer 30 may include a lens 34 or an opening on the housing which allows motion to be sensed, initiating the process of sanitizing. For example, if the sanitizer 30 is a door handle sanitizer, the approach of a person toward the door handle 22 may activate the sanitizer such that a person knows that the door handle has been sanitized through illustration of a green light or other mechanism. In addition, limiting the sanitization to a certain time period after motion allows conservation of the battery 62 and thereby less maintenance of the sanitizer 30. As the sanitizer 30 does not include a method of air moving the ions, such as a puff of compressed gas or other types of actions that are easily recognized as the sanitizer 30 being operational, the sanitizer 30 may include a visual or audible notice when the sanitizer 30 is functioning properly or a warning when function is impaired or the battery life is near the end of its life. In addition, a light pipe, such as a ring in the sanitizer in FIG. 13 may provide an indicator of proper function, such as a blue or green diode directed through the light pipe. To save energy, the diode may be pulsed, yet to a person viewing it, it looks constantly on. If the sanitizer 30 is attached to a moveable surface 10, such as a door 12, the controller 64 may include a simple accelerometer to detect motion of the illustrated door 12 instead of photo cell sensors or optical motion detectors. The accelerometer can detect motion of other types of fixtures 20 as well even if not mounted on a moveable surface 10. For example, a sanitizer 30 located inside of a refrigerator would still sense the motion of the door opening and closing even if it was not attached to the door of the refrigerator. An accelerometer is beneficial as compared to other motion sensors as it causes less battery drain. In addition, the sanitizer 30 may include a time delay with actuator. For example, upon swinging open the door 12 when someone enters a restroom, the accelerometer would be triggered which would cause the sanitizer 30 to activate for a specified time period, such as five minutes. Therefore, when the person leaves the restroom, the door handle 22 has been sufficiently sanitized. In addition, the opening of the door 12 upon exiting the restroom would also trigger the accelerometer and activation of the sanitizer 30 sanitizing the door handle 22 after the person leaves. Because the sanitizer 30 only functions during use of the restroom, battery life is conserved, while sufficiently sanitizing the desired object.

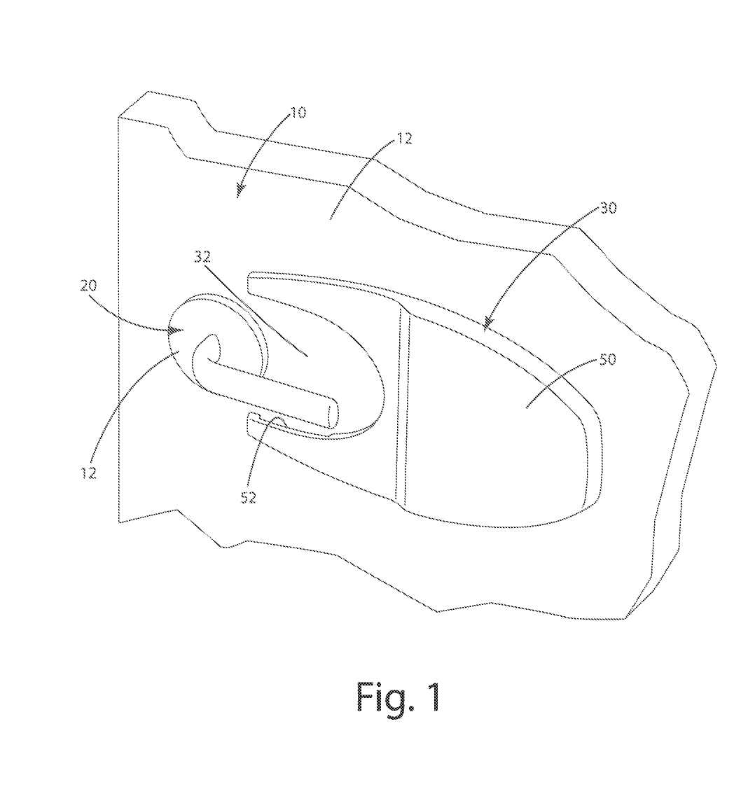

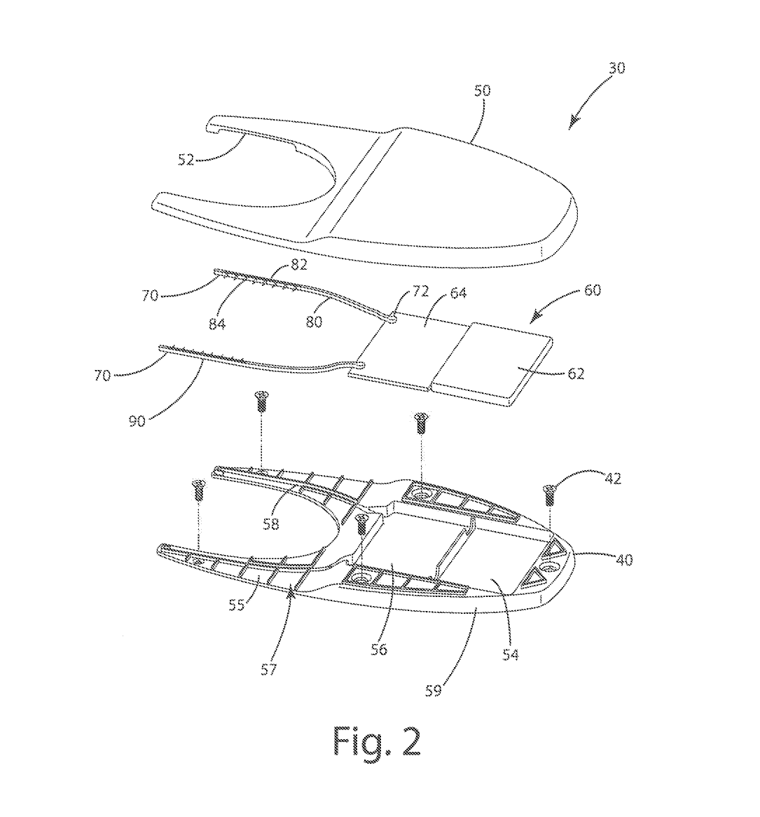

FIG. 1 illustrates a door 12 on which an exemplary sanitizer 30 is placed to sanitizer a fixture 20 such as the illustrated door handle 22. The illustrated sanitizer 30 as further provided in FIG. 2 is a fork design to surround the door handle 22 or fixture 20. As the electrodes 70 emit cold plasma, the electrical field may be used to move the ions and sufficiently sanitize the fixture 20 without the use of fans, CO₂ cartridges and the like. The fork design provides a fixture cavity 32 to surround a fixture 20 to be sanitized. A housing including the illustrated cover 50 is provided, and includes ion source openings 52 where the ions exit the sanitizer 30.

FIG. 2 illustrates a sanitizer 30 that uses a high frequency AC current applied to the electrode 70 containing the ion sources, which is also herein referred to as the ion electrode 80. The ion sources 82 are illustrated as small points but could be carbon fiber brushes or the like, which include many tips, each acting as an ion source 82 in place of the points 84. In the sanitizer 30, as illustrated in FIG. 2 that includes an ion electrode 80 having an applied AC current, a second electrode also may be referred to as the reference or ground electrode 90 is included and spaced some distance apart from the ion electrode 80 to prevent generation of ozone or arcing. As described in more detail later, as the AC current is applied to the ion electrode 80 with 1-80 Hz, preferably 5-70 Hz, more preferably 10-60 Hz frequency of alternating current is in turn driven by a transformer cycling at a high frequency, such as 20-400 kHz, on and off, typically in the higher end of the range. The ions both positive and negative leave the tips of the ion sources 82 on the ion electrode 80 and are pulsed outward until they cover the desired surface, such as the illustrated door handle 22 or fixture 20. In addition, in some embodiments, the ions emitted from the ion sources 82 may be drawn to the ground electrode 90 which helps them in the fork design, illustrated in FIG. 2, move across the fixture located between the two electrodes 80, 90. The high frequency AC sanitizer 30 generally has a voltage of 4000-15,000V, typically approximately 5,000-12,000V, and 7,500V most preferred when voltage is measured by the root mean square (RMS) method. The current output is typically 0.0002 amps and input will vary with the power source, typically 40-200 milliamps for most batteries. The input voltage may vary but is expected to be between 9-24V DC although 6-40V may be common.

As further illustrated in FIG. 2, the sanitizer 30 generally includes a base 40 in which the sanitizing apparatus, including a battery 62, controller 64, and electrodes 80, 90, is secured and a cover 50 placed over such components and secured to the base 40. The base 40 may include cavities for a battery compartment 54 and a controller cavity 56 as well as other cavities for receiving electrodes 80, 90, such as the illustrated electrode cavities 58. The electrodes 80, 90 are coupled to the controller 64 with a connector 72.

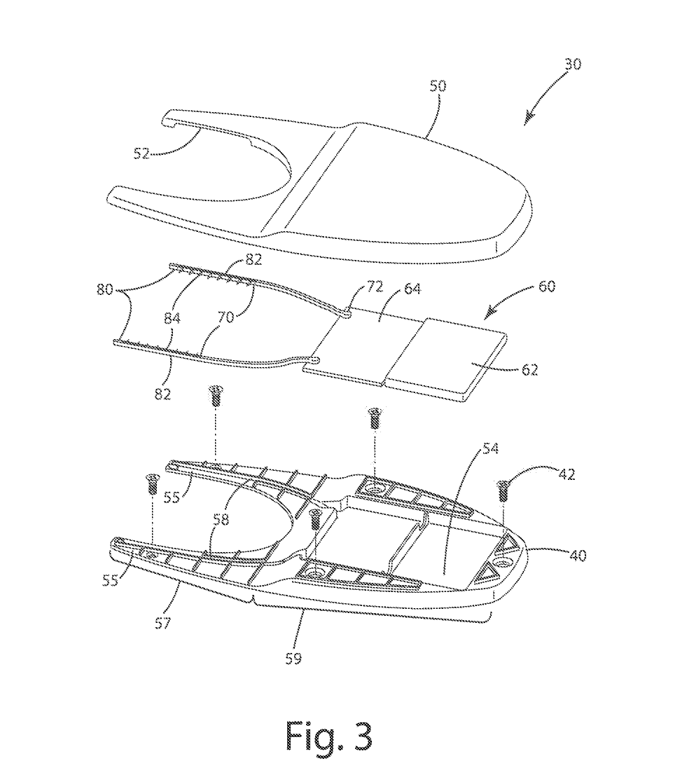

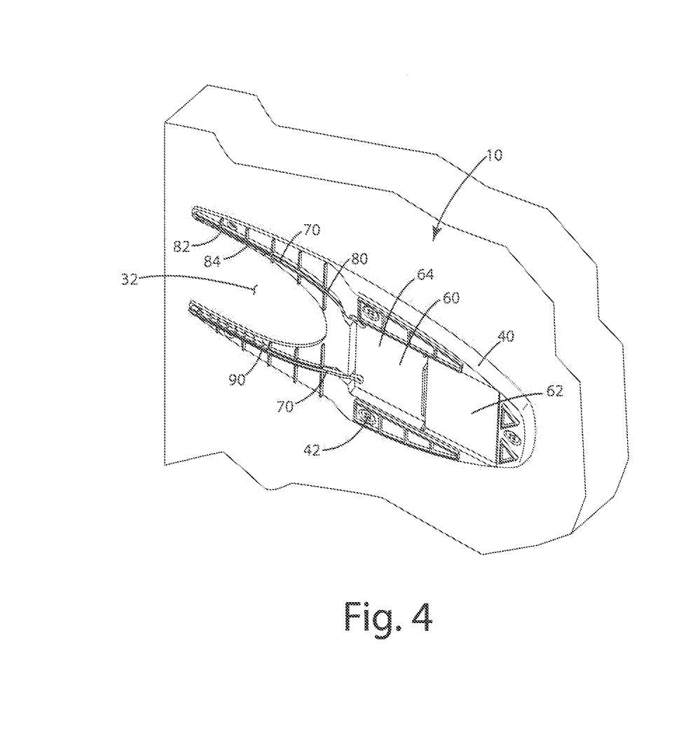

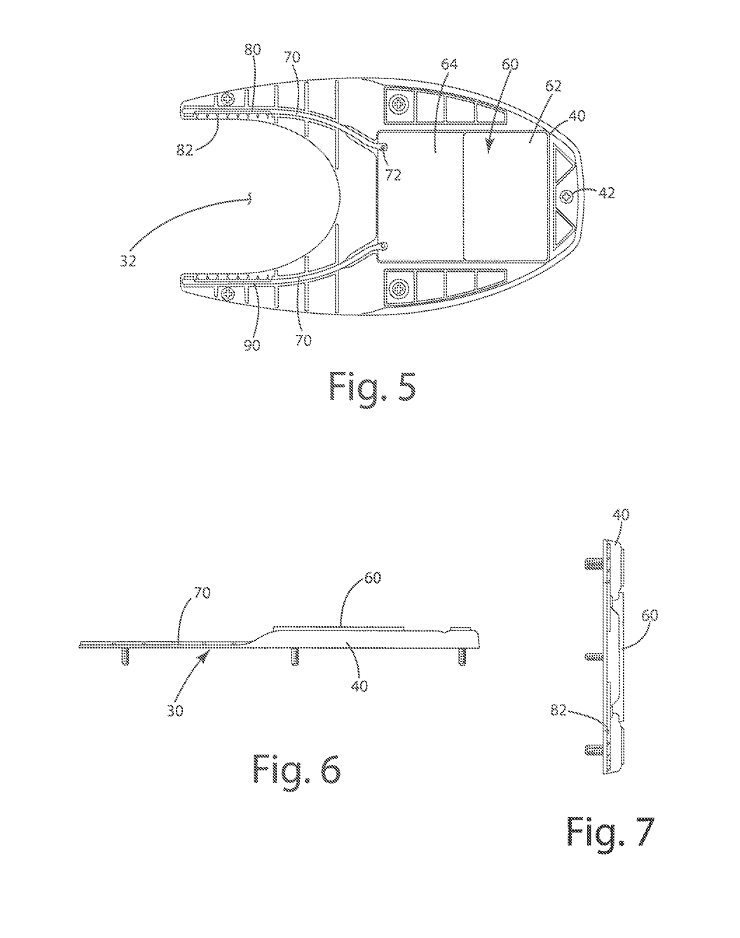

The sanitizer 30 illustrated in FIG. 3 includes two ion electrodes 80 and eliminates the reference or ground electrode 90 in FIG. 2. The use of two ion electrodes 80, each including ion sources 82, has a sanitizing apparatus, as provided in FIG. 3, that uses a pulsed DC of typically 3000-7500 volts typically 6000 volts is applied to each electrode 80 with, for example, one of the electrodes 80 emitting positive ions while the opposing electrode 80 emits negative ions. As such, the ions are drawn across the fixture 20 between the two ion electrodes 80 and the electrical field propels the ions toward the opposing electrode 80. A microprocessor controls the pulsed DC. The pulsed DC voltage may, for example, be produced by controlling a pair of transistors separately with pulse width (PWM) modulated signals from separate outputs of the microprocessor. Each transistor is used to energize the primary coil of a flyback transformer (e.g. one transformer and flyback transformer for the positive electrode and one transformer and flyback transformer for the negative electrode). When the transformer is switched off by the PWM signal from the microprocessor, the current in the primary coil and the magnetic flux drops. The voltage in the secondary coil becomes positive and current can then flow from the flyback transformer and create a voltage output at the electrode 80.

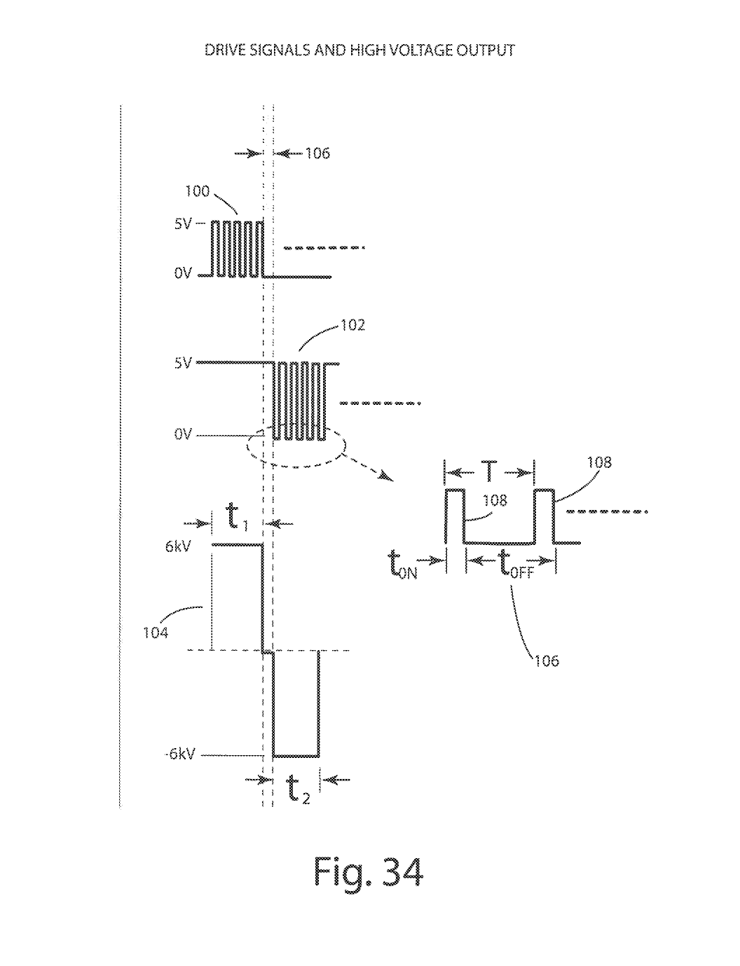

One electrode 80 of the sanitizer 30 of FIG. 3 may be connected to the secondaries of both flyback transformers so that a single electrode 80 produces both positive and negative ions from an AC output and the other electrode 80 may function as a ground. As shown in FIG. 34, a first drive signal 100 or PWM pulse train which will be described in more detail below drives the first flyback transformer to create the positive half of the AC output. Likewise, a second drive signal 102 or PWM pulse train drives the negative half of the AC output 104. The inventors have discovered that a “Dead Zone” 106 or period of time where both PWM pulse trains (i.e., first drive signal and second drive signal) are turned off is useful for efficient operation. Without a dead zone 106, the output from the flyback transformer driven by the first drive signal 100 may “shoot through” the flyback transformer circuit driven by the second drive signal 102 and vice versa. This may cause the outputs from each flyback transformer to somewhat cancel each other out. Adding a correctly sized dead zone 106 was shown to double the operating efficiency of the circuit. In other words, the voltage of the AC output 106 doubled while using the same amount of power.

Additionally, the level of ionization was found to increase significantly with the addition of a “Dead Zone” 106. It is thought that an abbacy change at a sharp discharge point 84 (needle point) causes emitted positive ions to combine and neutralize some of the negative ions that were emitted in the previous cycle and vice versa.

For electrical efficiency, the dead zone 106 must be a long enough time period for the previous half cycle output of the transformers energy to be dissipated and reach zero volts. The amount of energy that is initially stored in the flyback transformer by a tₒₙ pulse 108 shown in FIG. 34 and the transformer circuits characteristics (inductance, DC resistance and capacitance) determine the required duration of the dead zone 106. In one example, the dead zone 106 should be no less than 2 microseconds and no more than 20 microseconds.

For ion generating efficiency, the duration of the dead zone 106 is longer that what is required for electrical efficiency. The duration of the dead zone 106 for optimum ion generating efficiency also depends on the velocity of the air passing by the discharge point(s) 84. If the air is still (velocity=0) then a large dead zone 106 is required. If the velocity of the air passing over the discharge point(s) 84 is great, a smaller dead zone is required. The inventors have found a dead zone 106 of 50-100 ms is optimal. With high velocity air such as a high speed hand dryer (185 MPH) or the CO₂ powered door handle sanitizer smaller dead zone of 2-10 ms is optimal.

The first drive signal 100 is a pulse width modulated, PWM drive signal from the microprocessor to a circuit that produces the positive half of the AC output 104. The first drive signal 100 will be active while the second drive signal 102 is off. The first drive signal 100 is operated at a frequency between 20 KHz to 400 KHz depending on the characteristics of the flyback transformer being used. Ideally, a small flyback transformer with very low primary DC resistance and very low inductance is more energy and cost efficient and can be driven at a higher frequency. However, it has been found that the circuit works well with larger flyback transformers at the lower frequency range shown. The second drive signal 102 is similar to the first drive signal, except it drives the negative half cycle of the AC output 104.

The high voltage AC output 104 is shown in FIG. 34 as it relates to the two drive signals 100, 102 and the dead zone 106. Although, the AC output 106 is shown with a peak voltage of 6 KV, this can be varied from 2.5 KV to 12 KV by changing the PWM of the first drive signal 100 and the second drive signal 102.

The period of the drive signals 102, 104 is T. The period, T is inversely proportional to the frequency, f (T=1/f). The duty cycle is defined as the relationship between on time (tₒₙ) and off time (t_{off}) during one period (T). Because flyback transformers operating in discontinuous mode, (i.e. the current in the secondary of each flyback transformer is allowed to discharge completely to zero) the duty cycle should be less than 50%—meaning that off time is greater than on time. Typically, the duty cycle approaches 50% to achieve maximum voltage output. However, the inventors unexpectedly discovered that it is not necessary and even detrimental for the duty cycle to approach 50%. This is because it is necessary to utilize sufficient off time for the transformer circuit (transformer and voltage multiplier) to fully discharge before applying another pulse. In one example, it was discovered that a duty cycle of 10% resulted in maximum AC output 104 voltage. The duty cycle may be reduced as low as 2% to adjust the AC output 104 to its minimum.

The first drive signal 100 and second drive 102 signal may also be comprised of signals having different duty cycles. For example, if the duty cycle for the first drive signal 100 is 20% and the duty cycle for the second drive signal 102 is 30% a balance of more negative ions than positive ions may be achieved, which is beneficial for human wellness. Also, in an indoor environment with lower air quality, more negative ions may get “used up” and therefore, the negative ion output may need to be increased further compared to the positive ion output. In another example, if the air is passing through a duct that has a negative surface charge, (static electricity) more positive ions may need to be created as compared to the amount of negative ions being produced.

Of course, the electrode 80, 90 as well as the sanitizer 30 may be made in a variety of other configurations such that the electrodes 80, 90 may surround entry doors 12, restroom doors 12, kitchen doors 12, faucets, keypads, hospital fixtures, or any device that is touched on a regular basis such that it may include bacterial or other pathogens, which are undesirable and should be sanitized from the surface 10. In addition, electrodes 80, 90 can be built into various phone and tablet or computer cases, such as those used by doctors and hospitals to prevent the spread of infectious diseases. Electrodes 80, 90 may also be used proximate to other items receiving high frequency of touches such as vending machines, card readers, credit card payment devices and any other devices. Any surface 10 may be sanitized from refrigerator shelves and microwave turntables to kitchen countertops and desks and even the surfaces of food to prevent the growth of bacteria that spoils food.

The illustrated sanitizing apparatus 30 generally includes a battery and a control circuit such as the illustrated controller 64. The electrodes 80, 90, as illustrated, are formed of a conductive plastic material such as a conductive ABS material but of course could be formed of other conductive plastics such as a conductive polycarbonate or a blend of ABS and polycarbonate. In addition, the electrodes 80, 90 could be formed of metal including stainless steel, aluminum, nickel or other metals and metal alloys. Forming the electrodes 80, 90 of a plastic material allows molding of electrodes 80, 90 including, as illustrated in the Figures, molding the electrodes 80, 90 in place directly to the circuit board, specifically the controller 64. The present invention uses a conductive ABS material that has been doped with carbon but also could be doped with other materials, such as 15% stainless steel. Use of a conductive ABS allows a cost-effective material that is flexible and easy to assemble. Other cost effective conductive polymers include conductive polypropylene, doped with carbon, boron, or the like. The housing, including the base 40 and cover 50 is formed from a non-conductive material. The electrodes 80, 90 as illustrated are injection molded, although other methods may be used. To obtain the illustrated points 84, which are not possible with injection molding, given the size of the points, the dies are scored to create flash at the points, which creates the pointed surface the present invention uses to create the ions. The illustrated points 84 protrude about 4 mm from the electrode base, which is also about 4 mm wide and 1.6 mm thick, although other dimensions could be substituted. In the present invention, the ion sources 82 are generally spaced more than a ¼″ or 6 mm apart, but less than 2″ or 50 mm apart. It has been found that the pulse effect to drive the ions away from the ion sources 82 at less than ¼″ apart generally causes the ions to cancel each other out and at more than 2″ apart, the ions may not be applied as uniformly to the surface 10. In the illustrated embodiment, the ion sources 82 are spaced about ½″ or about 12.5 mm apart. The most effective range of spacing has been found to be about ⅜″ to 1″. The points 84 of the electrode 80 forming the ion surfaces are recessed in both sanitizers 30 by about 4-8 mm, typically about 6 mm. In addition, using a conductive plastic avoids potential corrosion of metal electrodes and many of the harsh environments where sanitizers 30 are desirable to be placed. For example, in a restroom, humidity as well as harsh cleaning supplies are regularly applied or incurred by fixtures 20, including the sanitizer 30 within the restroom and after a certain time period, even stainless steel may corrode.

The sanitizer 30 may be attached to a desired area through a variety of mechanisms, such as the illustrated fasteners 42. As assembled, it is desirable for the sanitizing apparatus to be unobtrusive and maintenance free as possible. Of course, as described above, the sanitizer 30 may be directly built into the fixture 20, appliance, or other surface 10.

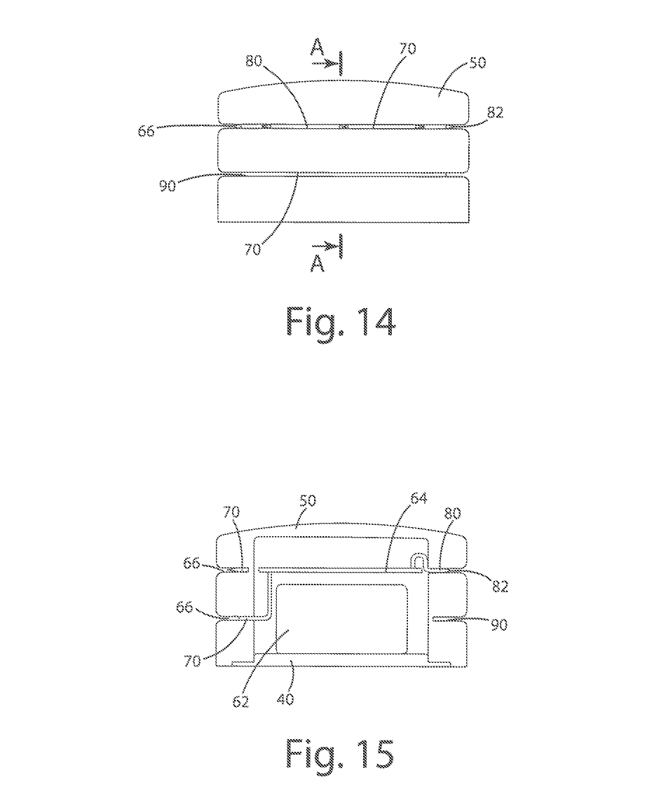

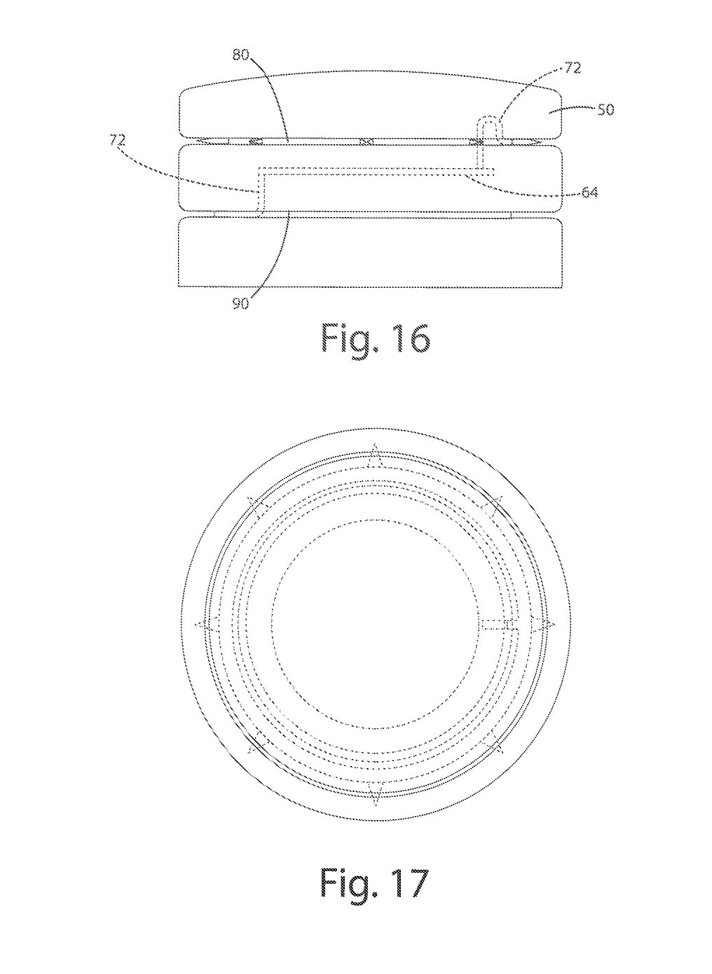

The sanitizer as illustrated in FIGS. 13-17 is specifically configured to provide a wide dispersal of ions such that the fixture 20 does not need to be centered between two electrodes 80, 90 as with the illustrated fork design. The illustrated sanitizer in FIGS. 13-17 is illustrated as having 360° of ion sources 82 but of course by removal of some of the ion sources 82 from the ion electrode, the coverage of ions may be reduced to less than 360°. In addition, the number of ion sources 82 shown on each ion electrode 80 may vary as well as the position or placement may vary depending upon the desired application. The sanitizer as illustrated in FIG. 13 generally includes a housing having a cover 50 and a base 40. An ion electrode 80 having ion sources 82 such as the illustrated points 84 extending out therefrom is illustrated further in FIGS. 20 and 21. A controller 64, a ground electrode 90 and a battery 62 may be assembled to the base and then covered with the cover for general protection. It has been found that use of the sanitizer as illustrated in FIG. 13 may provide sufficient generation and dispersal of ions across a six foot radius area from the sanitizer to substantially sanitize the surfaces 10 or at least reduce the number of pathogens and other infectious diseases on such surfaces 10. For example, a restroom, kitchen or other facility may include a number of these sanitizers secured to ceilings, countertops or walls, thereby providing substantially continuous coverage across the whole area to sanitize or reduce the number of infectious diseases on a majority of the proximate surfaces. The illustrated sanitizer in FIGS. 13-17 includes a ground electrode 90 and as such, uses a high frequency transformer to drive an AC current applied to the ion electrode to generate the ions at the ion sources 82. Of course, a pulsed DC version where the ground electrode 90 is swapped for an ion electrode 80 may also be used, but preferably would be placed in a setting experiencing air movement. In contrast, the configuration of the AC version as well as the method of operation allows the six foot ion dispersal range away from the sanitizer without air movement. Similar to the above, the electrodes 80, 90 also may be formed of a conductive plastic material such as a conductive ABS, although again, various other metals or alloys may also be used to create the electrodes 80, 90. The electrodes 80, 90 each include connectors 72 allowing for easy assembly to the controller 64, which is illustrated as a round disc in the Figures. Of course, the configuration of the sanitizer and individual components therein as illustrated in FIGS. 13-17 may vary depending upon the desired application. For example, while the controller 64 is illustrated as a round disc fitting nicely within the cover, a square circuit board may readily be used that fits within the cover and the outer size, shape, and configuration may vary depending on the application, but the relative placement of the electrodes 80, 90 and amount of recess of the electrodes 80, 90 will stay within the ranges described elsewhere in the specification. The controller 64 is expected to be sealed with epoxy or another material. The battery 62 as used in the sanitizer may be any type of battery 62, however a long-life battery such as a lithium ion battery is generally preferred. In some embodiments, the sanitizer may be hardwired into a facility or appliance power supply. The use of a lithium ion battery allows extension of the intervals between required maintenances and replacement of the battery, as compared to more traditional batteries. The illustrated sanitizer in FIG. 13 may be assembled through a variety of methods including where the cover is capable of being split into multiple pieces and snapped together or ultrasonically welded together with the electrodes fitting within the illustrated grooves 66 on the cover. In addition, the ion electrode 80 and ground electrode 90 may be formed with a small split on at least one side allowing expansion of the electrodes 80, 90 as they slide over the cover and then contraction as they fit within the specified and desired groove 66.

The grooves 66 on the cover are spaced about 10-15 mm apart and the recesses are about 14 mm deep, with the point 84 being recessed by 3 mm from the surface. The electrodes 80, 90 are closer on the round design illustrated in FIG. 13 than the fork design because the electrodes 80, 90 being recessed avoids arcing that would otherwise occur if the electrodes 80, 90 were spaced less than 20 mm apart on the surface of the cover. Therefore, the groove 66 allows closer spacing of electrodes 80, 90 and a smaller package to the sanitizer. However, the depth of the groove 66 relative to the spacing of the grooves 66 is also important as too deep of a groove 66 may prevent sufficient expulsion of the ions from the groove 66. As the electrodes 80, 90 are more recessed in the grooves 66, the spacing of the grooves 66 may shrink and as the electrodes 80, 90 approach the surface of the cover, the spacing of the grooves 66 increases to prevent arcing and ozone generation.

The battery 62 may also be rechargeable, and the sanitizer could include a USB port or other input that could provide charge to the battery 62. In addition, the device may include Bluetooth or Wi-Fi to allow control of the device with smartphones, computer, tablets, and the like, or for a person to check the status of all devices within a facility or within a given range. Control over the voltage output, and as such amount of ions generated as well as battery life could be controlled. Any inputs, such as a power supply input, USB input and the like may be covered to prevent liquid intrusion, such as if a sanitizer was used on a kitchen counter.

For use in fixtures 20 and appliances, the ion generator or sanitizer 30 may be included as part of the fixture 20 or appliance, with metallic portions of the fixture 20 or appliance forming the ground plane. One exemplary configuration is for the ion generating electrode 80 with its points 84, brushes or other ion sources 82 to be located a recessed area to avoid anyone coming into contact with the sharp points 84. An insulator may be disposed between the ion electrode 80 and the metallic or conductive plastic areas on the housing or surfaces of the fixture 20 or appliance. The ion electrode 80 and ground plane forming the ground electrode 90 would be spaced as provided below. The ion generating electrode 80 is electrically insulated from the ground electrode 90. While the sanitizer 30 in the Figures illustrates a specific electrode acting as the ground electrode 90 or ground plane, objects on the device or the sanitizer 30, or as described above with the appliances or fixture 20 could form the ground plane. For example, to sanitize proximate to the kitchen sink or faucet, one of the sink or faucet could be a ground plane for the ion generating electrode 80. As it is a ground plane, and naturally grounded through the plumbing, the ion generator could be configured to attach the ground electrode 90 to the metal pipes of the plumbing or metal fixtures of the plumbing. Therefore, the faucet is the ground, and a ring or plate could extend under the faucet or around the faucet, such as a plastic insert around the faucet and includes in a recess, the ion generating electrode 80. It is generally preferable to recess the ion generating electrode 80 to prevent contact with the ion sources 82 on the ion electrode and to create a torturous pathway so minimize packaging around the ion electrode 80 and spacing required to the ground electrode 90. In addition, the recess may allow sufficient distance from metallic portions of the surface 10, fixture 20, or appliance acting as a ground plane or ground electrode 90.

It is important to note that the ion generator or sanitizer 30 generally includes a large resistor such as a 50 mega ohm protection resistor 120 in the present invention, which limits the current as a safety feature and limits it to micro amps of current. The ion generator could also be used in a shower to prevent growth of mold, bacteria and other pathogens in a shower, particularly public showers or enclosed showers where humidity stays present and promotes undesirable growth. Also, the more humidity that occurs in a shower the more effective the ion generator is at generating ions and therefore more effective at greater distances.

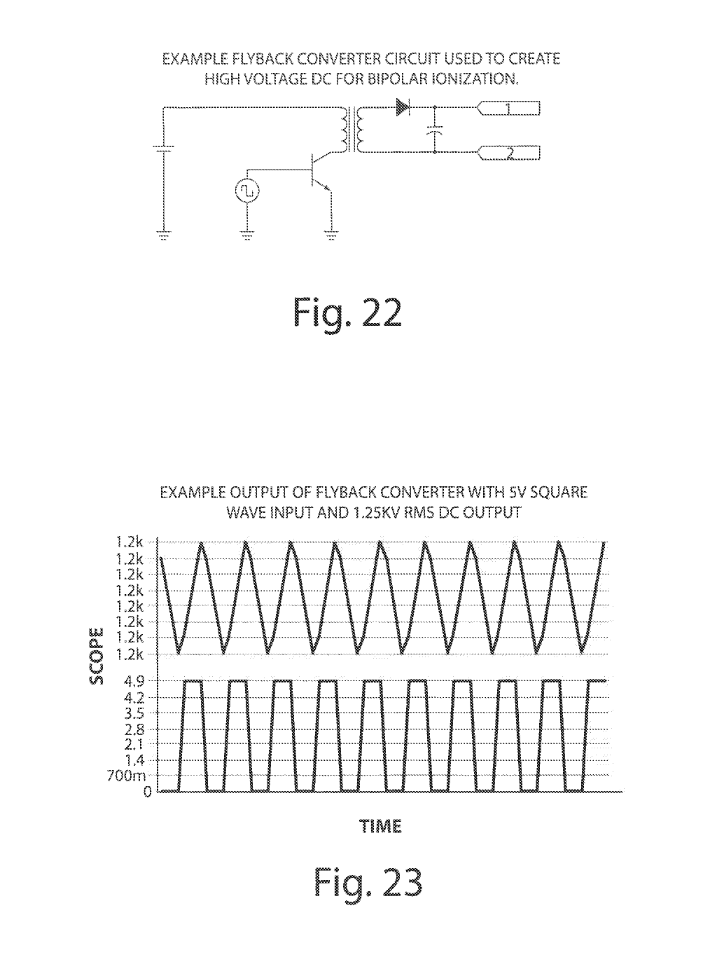

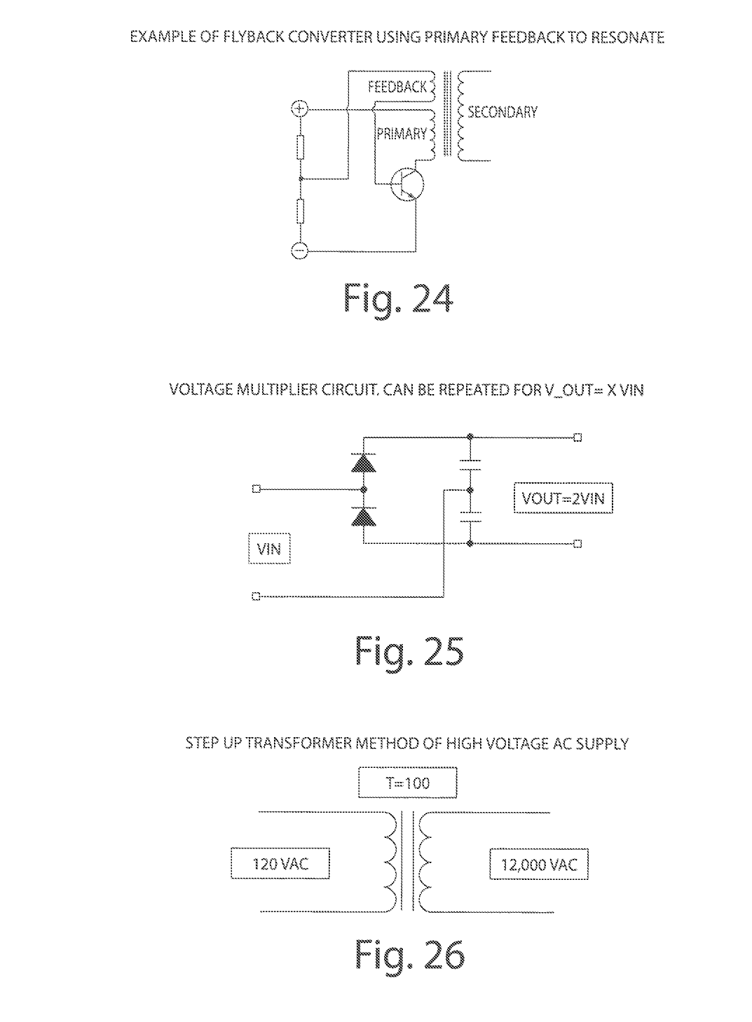

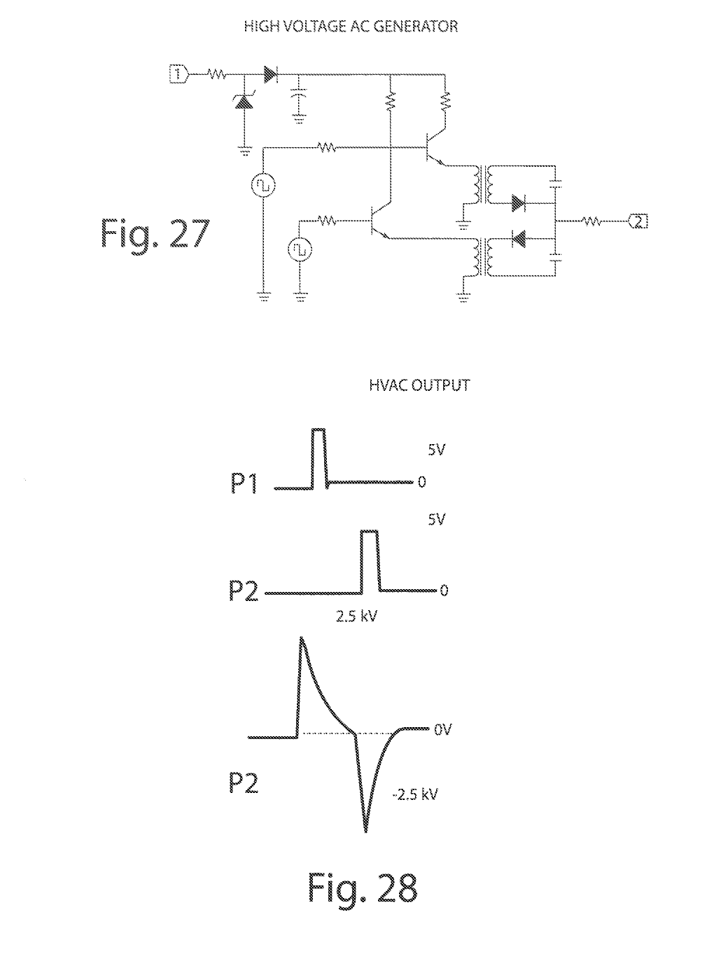

As discussed above, high voltage power supplies are commonly used for cold plasma generation. Many ionizers or ion generators use a high voltage DC power supply which have traditionally been considered the most compact and economical. One typical method to create the high voltage charge for producing ions is to use a flyback converter using primary feedback to resonate. Variation of this circuit is shown in FIGS. 22 and 24. While simple and inexpensive, this approach has several disadvantages. First, the output voltage is not regulated and as such varies greatly while the circuit warms up. For some flyback convertors, the output may take up to 20 minutes for the output to stabilize. Even once warmed up, the output will still vary greatly with temperature or the input voltage, which is often unregulated.

In applications in close proximity to humans, it is desirable to have a well-regulated voltage output to ensure proper production of ions and to avoid production of compounds harmful to humans. For example, if the output drifts too high, arcing between the discharge points can occur causing a corona discharge that may produce ozone, which has been found to be harmful to humans if the amount of ozone exceeds certain threshold levels. Arcing or corona discharge may also occur between the discharge points and the metal of ductwork in heating, ventilation, and air conditioning systems, as well as appliances and fixtures. The sound caused by the arcing may be audible concern people in the proximity. Finally, this arcing can cause the ion generator to fail by melting conductors or otherwise damaging or degrading nearby components.

A regulated DC pulse provided to the primary winding of the flyback transformer may eliminate some of the above described issues. For example, the pulse may be controlled with a timing chip such as the LM555 or a microcontroller as illustrated in FIG. 23.

Another approach to create a high voltage is a voltage multiplier circuit, illustrated in FIG. 25. The input voltage can be AC or DC. However, the output will be DC, and over time, a DC output tends to collect dust, which reduces its ability to produce ions.

The flyback transformer can also be combined with a voltage multiplier to generate a higher output voltage. This approach is typically required when the secondary winding of the flyback transformer reaches the limits of its dielectric strength and cannot output a higher voltage without failure.

As discussed above, most ion generators require a means of propulsion such as compressed air or CO₂ to move the ions away from the ion source, however, the inventors have surprisingly found that a high voltage AC ion generator is capable of moving the ions away from the ion sources if properly configured and operated within certain operational ranges. In addition, the AC version described herein actually is an improvement in dispensing ions without separate means of propelling ions away from the ion sources as compared to traditional DC ion generators that use two electrodes, each have any opposing charge. The ion generator of the present invention creates more ions, uses less power, particularly less power from battery packs, and expels the ions a greater distance from the ion electrode without the need for additional propulsion, such as compressed gas in sanitizers. More specifically, an alternating current (AC) high voltage source has been found to be ideal for ion generators particularly when compared to traditional DC sanitizers. However, it should be noted that the DC sanitizer with the fork design overcomes the limitations of DC sanitizers particularly with regards to the fixture cavity as illustrated in FIGS. 1 and 3. One unique feature of the present invention is that the AC high voltage ion generator only requires one discharge electrode 80 which may have one or more points 84, not two discharge electrodes, otherwise referred to as ion generating electrodes 80, yet can function as a bipolar ion generator that generates both positive and negative ions. This single discharge point or single ion electrode 80 (which can have multiple discharge points 84 along the electrode as illustrated) can alternate between creating positive and negative ions. The inventors have found that this surprisingly yields the following advantages: (1) only one discharge point required to create both positive and negative ions, although a ground electrode 90 may be still used to create a ground plane; (2) by alternating polarity of the single discharge point or electrode 80, it is far less likely to be contaminated with dust and will therefore have greater service life, because dust particles or other contaminants are attracted to the discharge point or electrode when it is positively charged will be repelled when it is negatively charged and vice versa; and (3) the use of AC high voltage ion generator can deliver higher concentrations of positive and negative ions at a greater distance from the discharge point(s) 84. The fact that the ion generating electrode 80 does not attract dust like the positive electrode of prior DC ion generators allows a longer service life and maintains operational performance closer to original specifications over the service life of the ion generator as the dust interferes on a DC ion generator with the generation of the positive ions. However, with regards to the illustrated DC sanitizer, the inventors have found that a burst of higher discharge may burn off dust particles, and while such a discharge may create ozone, the duration would be so short and so infrequent that barely any ozone would be created and would not noticeably add to the level of ozone in the proximity of the sanitizer and be under all applicable rules or regulations regarding the discharge of ozone. In addition, typically it was believed that to generate sufficient ions, at least two electrodes having opposing charges were required, or at a minimum, a sacrificial electrode was required. In the present invention, no sacrificial electrode is required, the single electrode 80 generates all of the ions, and it is believed that the alternating current and resulting alternating production of positive and negative ions generates a pulse effect, similar to the ripples in water when an object is dropped in that as small waves expand outward. In the present invention, the pulsing creates waves that cause the ions to travel away from the ion generating electrode 80.

While the ion generator 110 of the present invention uses high voltage AC, which the stepped up or higher voltage AC is usually created using a step-up transformer, the step up transformer is not preferred as discussed below. In a step up transformer, a low voltage AC supply is supplied to the primary side of the transformer. The step-up transformer provides an output voltage that is equal to the input voltage multiplied by turns ratio of the step up transformer. For example, a transformer with 10 turns on the primary and 1,000 turns on the secondary has a turns ratio of 100 (T=100). If 120 VAC were applied to the input, the output voltage would be 12,000 VAC. While such a solution is simple and effective method for high voltage AC supply, it suffers from poor electrical efficiency, high cost, and large size.

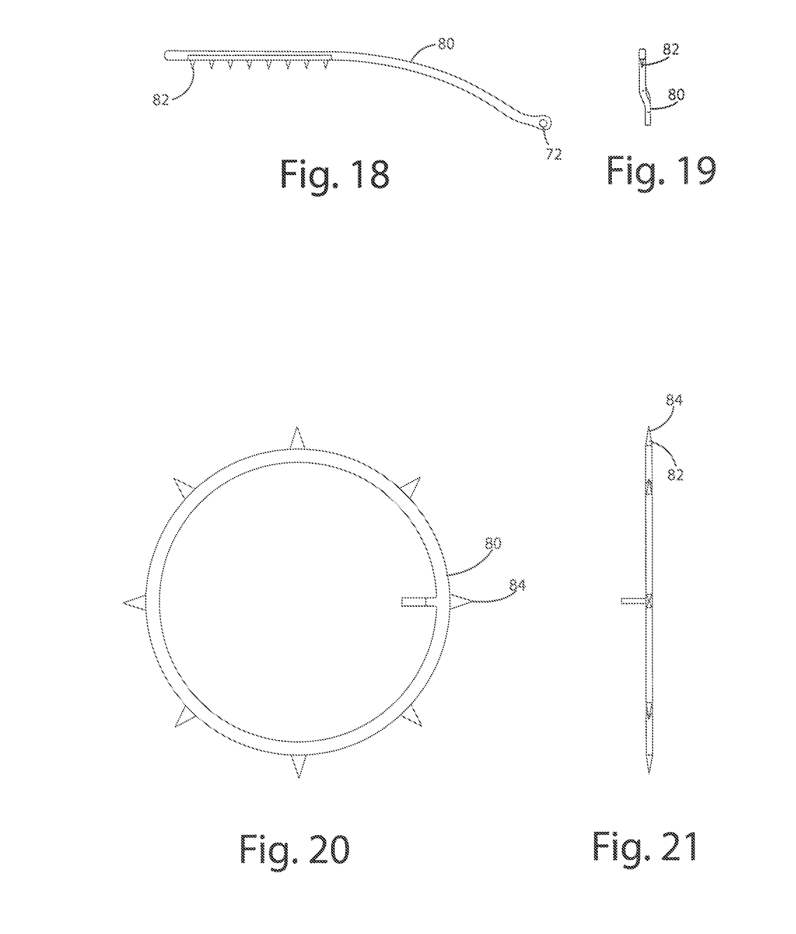

Therefore, as stated above, the present invention can use a step up transformer, however the inventors have found it preferable to reduce the size of the packaging and the power loss due to heat generation. Therefore, the present invention creates high voltage AC for a single discharge point bipolar ionizer or multiple discharge points that experience the same positive or negative charge at the same time. The present invention uses two flyback transformers 140, 142 resulting in a design which does not require the size, cost, weight, or energy consumption of a step-up transfer design. Further, the proposed design can accept a variety of AC or DC inputs to create the high voltage AC output. A simple potentiometer (pot) can be provided to allow adjustment of the high voltage AC output for different applications. The range of AC output required to generate ions may vary, however the inventors have found that a minimum of 3000V peak to peak (e.g., +1500V to −1500V), preferably 4000V peak to peak, and more preferably at least 5000V peak to peak, but in no event more than 12,000V peak to peak, preferably less than 8000V peak to peak and more preferably less than 7500V peak to peak. The above voltages may vary depending on spacing and are set for the ion generating electrode 80 to be spaced between about 2 cm and 5 cm (¾″-2″) from the ground plane or ground electrode 90. As such, for these spacings to avoid creating of ozone, the voltage ranges are critical, and as such, typically as the electrodes are placed in closer proximity the lower end of the ranges above is preferred and as the spacing increases the higher end of the above voltage ranges is preferred. In addition, beyond strictly the distance, if the distance is a torturous pathway between the ion electrode 80 and the ground electrode 90, such as the illustrated puck design in FIGS. 13-21, the voltage may be run at a higher voltage than if both of the electrodes 80, 90 were placed on the same surface 10 with no intervening obstructions as the latter would be more likely to arc or create ozone. As it is best to balance power consumption and the amount of ions generating a range of voltage for the ion generating electrode to be spaced 2-5 cm from the ground electrode is typically 3000-7500V peak to peak, and preferably 4000-6000V peak to peak, and more preferably 5000-6000V peak to peak. As stated above, all of the voltage measurements provided are RMS voltage. As stated above, the present invention uses two flyback transformers 140, 142, one to create the positive half of a high voltage AC output and the other to create the negative half of the high voltage AC output. The two outputs are combined into a single high voltage AC output. A micro controller or microprocessor 144 is used to switch the transformers 140, 142 in a stable manner. The use of two flyback transformers 140, 142 that are switched also improves the output of the ion electrode 80, because the system is almost immediately at full power, maximizing production of the ions at the ion electrode 80, whereas a flyback transformer utilizing feedback from a primary or secondary coil to create a resonator does not stabilize to full power for a long period of time. FIG. 28 clearly illustrates the immediate spike in voltage over time against the square wave of the flyback transformers 140 and the slow drop off in voltage to the ion electrode 80 after the square wave has ended and then the immediate opposite jump in voltage as the square wave of the other flyback transformer 142 is applied. As the microcontroller 144 switches back and forth, the pattern is repeated. As illustrated in the Figures, a 5V input is provided and 2500 V output is then provided. Of course other voltages, both output and input may be configured and provided.

The cycle rate between series of positive and negative peaks or drive signals 100, 102 (i.e. to provide the high voltage AC output) is preferably at least 10,000 Hz, and more preferably at least 25,000 Hz, and for the illustrated exemplary configuration in the Figures, the ion generator 110 operates at about 100,000 Hz, which provides the best balance of generating ions, low cost, and low power requirements. FIG. 34 illustrates example drive signals 100, 102 and resulting high voltage AC output. It has been found that even with such quick cycling, the ions are sufficiently generated and the present invention typically uses 75,000-100,000 Hz frequency rate. It is important to note that the exemplary configuration of the present invention does not use a 60 Hz cycle rate and more importantly that the present invention using an ion generator 110 operating at 100,000 Hz and 3000-7500V, preferably 5000-6000V peak to peak is operating at what many skilled in the art consider unstable and attempt to avoid. However, the inventors have surprisingly found that these parameters offer the best generation of ions, particularly when measured against the power consumption of the ion generator 110 where it is desired to maximize battery life.

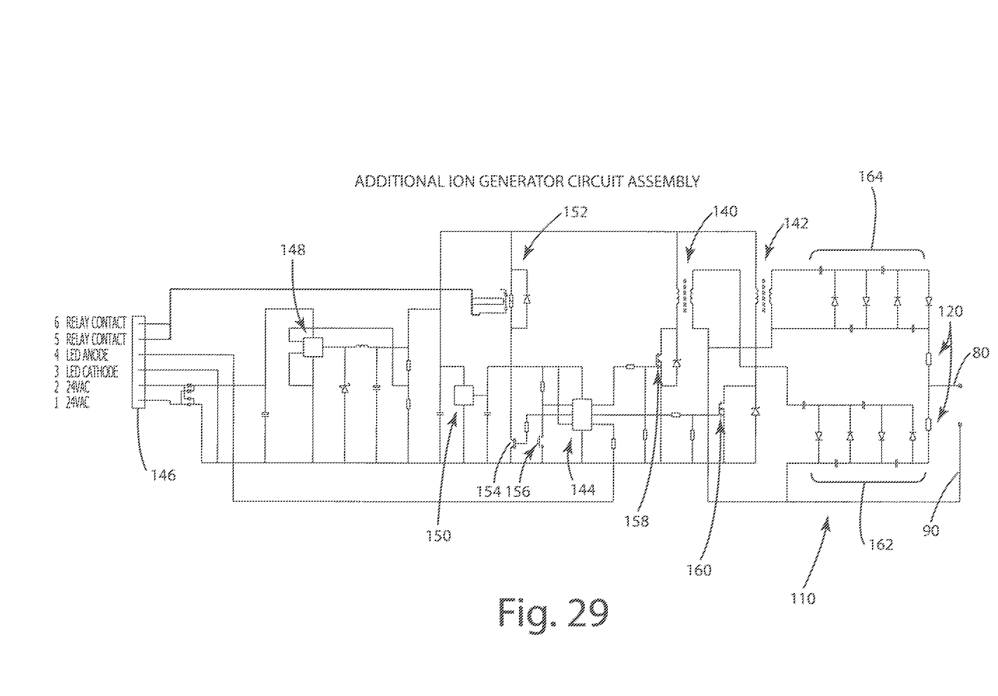

As shown in FIG. 29, an additional ion generator 110 includes a circuit assembly for ion generation which differs from that of the exemplary ion generator 110 discussed above. The circuit assembly includes a wiring connector 146 having a pair of relay terminals, a light emitting diode (LED) anode terminal, an LED cathode terminal, a 24 VAC positive terminal, and a 24 VAC negative terminal (ground). The LED cathode terminal is connected directly to the 24 VAC negative terminal.

A switching regulator 148 having an output is electrically connected to the 24 VAC positive terminal and the 24 VAC negative terminal of the wiring connector 146. An input capacitor is connected across the 24 VAC positive terminal and the 24 VAC negative terminal to prevent large voltage transients input to the switching regulator 148 from the 24 VAC terminals. The switching regulator 148 outputs a lower voltage on the output connected to the 24 VAC negative terminal (ground) through a Schottky diode and connected to an inductor which is also connected to an output capacitor tied to the 24 VAC negative terminal. The Schottky diode provides a return path for the inductor current when the switching regulator is deactivated. Two resistors are connected in parallel to the output capacitor and a feedback line is connected between the output resistors and to the switching regulator. Although, the switching regulator 148 of the currently discussed ion generator 110 is a LM2576 manufactured by ON Semiconductor, it should be understood that other ion generators 110 may use different switching regulators 148, or may not use a switching regulator 148 at all.

A positive voltage regulator 150 having an output is connected to the output resistors of the switching regulator 148 and to the 24 VAC negative terminal for regulating the voltage of the output from the switching regulator 148. A capacitor is connected in parallel with the resistors. An additional capacitor is connected between the output of the positive voltage regulator 148 and the 24 VAC negative terminal (ground). The voltage regulator 150 of the currently discussed ion generator 110 is an L78L05 manufactured by ST Microelectronics, as with the switching regulator 148, it should be understood that other ion generators may use different positive voltage regulators 150, or may not use a positive voltage regulator 150 at all.

A relay 152 having a coil is electrically connected to the relay terminals of the wiring connector 146 and to the output of the switching regulator 148. A reverse biased diode is connected across the coil of the relay 152 to allow transient voltages generated when the voltage is removed from the coil to be dissipated in the resistance of the coil wiring. The relay 152 of this ion generator 110 is a G5V-1 manufactured by Omron, it should be appreciated that other relays 152 may be used and that other ion generators may use relays 152 with different characteristics, or may not use a relay 152 at all.

A microprocessor 144 having a plurality of input/output (I/O) terminals is connected to and powered by the output of the switching regulator 148. A bipolar transistor 154 having a gate input is connected to the coil of the relay 152 which is also tied to the to the output of the switching regulator 148. The bipolar transistor 154 is also connected to ground (24 VAC negative terminal). The gate input of the bipolar transistor 154 is connected through a resistor to one of the I/O terminals of the microprocessor 144. The microprocessor 144 can energize the coil of the relay 152 through the I/O terminal connected to the gate input of the bipolar transistor 154. Another I/O terminal of the microprocessor 144 is connected through a resistor to the LED anode terminal of the wiring connector to control an LED. A separate I/O input is connected to the output of the switching regulator 148 through a resistor and tied to ground by a switch 156. Two other I/O terminals of the microprocessor 144 include a first pulse width modulated (PWM) output and a second PWM output. The microprocessor 144 utilized for the currently discussed ion generator 110 is a PIC 12F609 manufactured by Microchip, however, it should be understood that other microprocessors 144 may be substituted.

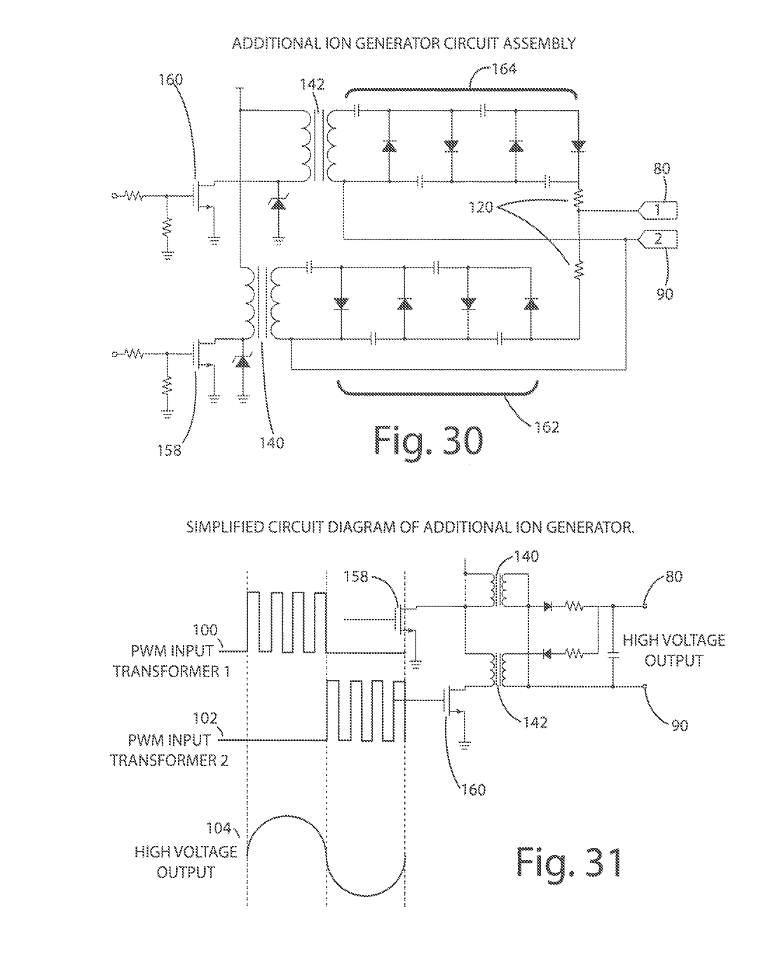

As shown in FIGS. 29 and 30, the first PWM output of the microprocessor 144 is connected to a first switching transistor 158 through a series resistor and a resistor connected to ground. A Schottky diode is connected across the first switching transistor 158 for preventing any overvoltage across the first switching transistor 158. In a similar fashion, the second PWM output of the microprocessor 144 is connected to a second switching transistor 160 through a series resistor and a resistor connected to ground. A Schottky diode is connected across the second switching transistor 160 for preventing any overvoltage across the second switching transistor 160.

A first flyback transformer 140 having a primary winding and a secondary winding is connected to the first switching transistor 158. More specifically, the primary winding of the first flyback transformer 140 is connected to output of the positive voltage regulator 150 and to the 24 VAC negative terminal (ground) through the first switching transistor 158. Thus, the first switching transistor 158 can control the amount of current through the primary winding of the first flyback transformer 140 in response to the first PWM output of the microcontroller or microprocessor 144.

A second flyback transformer 142 having a primary winding and a secondary winding is connected to the second switching transistor 160. Specifically, the primary winding of the first flyback transformer 142 is connected to output of the positive voltage regulator 150 and to the 24 VAC negative terminal (ground) through the second switching transistor 160. Therefore, the second switching transistor 160 can control the amount of current through the primary winding of the second flyback transformer 142 in response to the second PWM output of the microcontroller 144.

Each flyback transformer 140, 142 in the circuit assembly is controlled by the PWM outputs to energize the primary coils of each flyback transformer 140, 142. When the switching transistors 158, 160 are switched off by the PWM outputs, the respective current and magnetic flux drops in the primary winding. The voltage in the secondary winding of each flyback transformer 140, 142 becomes positive, and current is allowed to flow from the transformer 140, 142, generating a high-voltage peak in the secondary winding.

A first output section 162 is electrically connected to the secondary winding of the first flyback transformer 140 for amplifying the high-voltage peak from the first flyback transformer 140. Similarly, a second output section 164 is electrically connected to the secondary winding of the second flyback transformer 142 for amplifying the high-voltage peak from the second flyback transformer 142. Each output section 162, 164 includes a multiplier bridge comprising a plurality of capacitors and diodes arranged in a ladder configuration. The secondary winding of the first flyback transformer 140 is also electrically connected to a reference terminal or ground 90.

Flyback transformers generally can be operated in a continuous mode or a discontinuous mode. In the continuous mode, some energy is allowed to be stored in the transformer at all times. In a discontinuous mode, wherein the current in the secondary winding is allowed to discharge completely so that there is no longer any energy stored in the transformer. Although the flyback transformers 140, 142 of the present invention are operated in the discontinuous mode, it should be appreciated that in other ion generators, the flyback transformers 140, 142 may be operated differently than disclosed herein.

Each of the output sections 162, 164 are connected to a single emitter or ion electrode 80 through a protection resistor 120 (FIG. 29). The emitter 80 is disposed adjacent to the reference terminal 90. Positive and negative ions may be generated by the circuit assembly between the emitter 80 and the reference terminal 90. It should be appreciated that the instead of a reference terminal 90, the emitter 80 may instead be disposed near another ground such as “earth” ground, or a nearby metal structure (e.g., metal faucet to be sanitized by the ions being generated or ductwork in a heating, ventilation, and air conditioning system). One advantage to using “earth” ground is that positive and negative ions are more prone to spread everywhere, which may be desirable for some applications. The protection resistors 120 of the currently discussed ion generator 110 are 10 Mega Ohm resistors, however it should be understood that the impedance of the protection resistors 120 may be selected depending on the particular application of the circuit assembly and may even be excluded in some instances. The protection resistors 120 limit the current as a safety feature (e.g., if a person happens to touch the emitter). It should be recognized that an emitter 80 may have multiple points 84 or ion sources 82.

The combination of the first flyback transformer 140 with the first output section 162 creates one half of a high voltage AC output (e.g., positive portion of a sine wave) and the combination of the second flyback transformer 142 with the second output section 164 creates the other half of the high voltage AC output 104 (e.g., negative portion of a sine wave). In the currently discussed ion generator 110 and illustrated by FIG. 31, the first PWM output is generated by the microprocessor 144 at a rate of 50 kHz for approximately 16.7 ms to produce the first half of a sine wave and then the second PWM output is generated by the microprocessor 144 at a rate of 50 kHz to produce the second half of the sine wave. The two outputs are combined into a single high voltage AC output 104. This ion generator 110 produces a high voltage alternating current (AC) output 104 at approximately 60 Hz. Although the PWM output of this ion generator 110 is approximately 50 kHz, other PWM frequencies may be chosen, such as between 30 kHz and 400 kHz. The size of the flyback transformers 140 that is required is dependent on the frequency which it is driven at by the PWM outputs (i.e., faster frequency allows the use of a smaller flyback transformer). The output 104 of high voltage AC is advantageous because only one emitter 80 is required as discussed above. Additionally, by alternating between positive and negative ions, less cleaning is necessary as described above with the exemplary ion generator.

The inventors have discovered that approximately 0.1-100 Hz is a desirable frequency range (preferably 10 to 60 Hz) for the high voltage output. Because the emitter 80 is alternating between positive ions and negative ions, the ions must be given a sufficient amount of time to travel away from the emitter 80 before an ion with opposite polarity is emitted next. This output frequency could also be adjusted based on the velocity of air passed the emitter 80 (i.e. slower moving air would require lower frequency).

Because of the use of the microprocessor 144 of the currently discussed ion generator 110, it is possible to independently adjust the first PWM output independent of the second PWM output. Consequently, the amount of positive ions as compared to the amount of negative ions being generated may be independently adjusted. This adjustment could even take into account feedback from a sensor or ion counter which senses the ionization of the environment in which the ion generator is operating. It is believed that humans prefer an environment that includes slightly more negative ions. For this reason, it may simply be desirable for the ion generator 110 to produce more negative ions. The separate ion counter can be used by placing in the living space to monitor the number of positive and negative ions present. Typically, the number of positive ions is greater that the number of negative ions. Feedback from the ion counter can be used to change the output 104 of the ion generator 110 to rebalance the ions in the living space to healthy levels.

In order to accomplish the balance of negative or positive ions, the on time or duty cycle of the PWM outputs may also be adjusted by the microprocessor 144 to change the voltage level at the emitter 80. The voltage level may be adjusted for other reasons such as, but not limited to humidity, level of ionization, air velocity, or other properties of the air adjacent to the emitter 80. For example, air that has been ionized is more conductive. Another factor affecting the voltage level that may be used is the distance between the emitter 80 and a ground (e.g., “earth” ground) or between separate emitters 80. By providing a consistent fixed distance between the emitter and ground, the output of the ion generator 110 can be made more consistent. It is intended that the ion generator 110 of the present invention does not produce ozone as discussed above. Ozone may be created when there is an arc, therefore the voltage may be adjusted to prevent arcing and the production of ozone. A configuration of the present invention also includes a button that is connected to the microprocessor and can be used to command an increase or decrease in the voltage level in incremental steps (e.g., 3.5 kV, 4.5 kV, 5.5 kV, 6.5 kV). The button could be used by a consumer to adjust the voltage output, or by an installer of the ion generator. Holding down the push button for a few seconds places the device in program mode. An LED connected to the LED terminals of the wiring connector will first blink once for voltage setting one, twice for voltage setting two and so on. One example has four voltage outputs to select from 3,500, 4,500, 5,500, and 6,500 volts. Releasing the switch will select the voltage that corresponds to the number of times the LED blinks.

Many ion generators require the use of feedback to sense arcing or conditions which indicate that arcing is occurring at the emitter. Unstable output voltage can lead to arcing. Because of the stable high voltage output 104 of the present invention, no feedback regarding arcing of self-discharge is necessary. Detection and feedback of self discharge adds additional components and complexity to the ion generator 110, therefore the overall cost of an ion generator 110 can be reduced if feedback of self discharge is avoided. It should be understood that self-discharge detection could however be implemented in the present invention if self discharge requirements are more stringent. One possible way to detect self discharge would be to monitor the output voltage.

As mentioned, the present invention described above are less prone to contamination due to the emitter 80 alternately emitting positive and negative ions causing so that dust particles or other contaminants are attracted to the emitter or ion electrode 80 when it is positively charged will be repelled when it is negatively charged and vice versa. However, another configuration of the present invention includes a separate cleaning mode which intentionally creates a corona discharge to vaporize dust or contaminants on the emitter 80. Other ion generators may intentionally create ozone for short periods of time to help clean the emitter or area around the emitter 80.

In a separate ion generator for a heating, ventilation, and air conditioning application, multiple cold plasma generators are assembled together on an extruded mounting bar. The mounting bar can be cut to the required length and an appropriate number of cold plasma generators or emitters are installed on the bar. It is desirable to have an ion generator with sharp discharge points along a variable length for many applications including a faucet sanitizer, door handle sanitizer, and needlepoint systems for heating, ventilation, and air conditioning. One approach has been to use carbon brushes. Each carbon brush has to be electrically connected via a wire which requires many separate parts including electrical connections and a housing. This is expensive and will not fit into small spaces. If a DC high voltage source is used, two of these assemblies are required doubling the cost and the space requirements. Another approach is to press fit stainless steel needled into a plastic dielectric pieces and devise a conductive piece to connect the needles. An additional housing is also needed. A similar approach is to injection mold conductive plastic pieces with sharp discharge points and connect with a conductive piece and a housing. These function well be are limited to being manufactured to a specific length that cannot be adjusted in the field (cut). Finally, another approach is to cut a flat sheet of stainless steel into a shape with multiple discharge points. An insulated housing is required. This may be seem more cost effective and use less space. However, care must be taken to radius every sharp edge except the sharp tips of the discharge points to prevent leakage that will damage the insulating housing and also ensure that the discharge points are producing ions at the desired level. Finally, the unit cannot be cut to length to fit a specific application. Cutting the device to a shorter length leaves sharp edges that will cause plasma discharge (leakage). Covering the sharp edges with an insulator will only result in the discharge damaging the insulator, and leaking ions where not desired, which reduces ion output where desired from the ion sources.

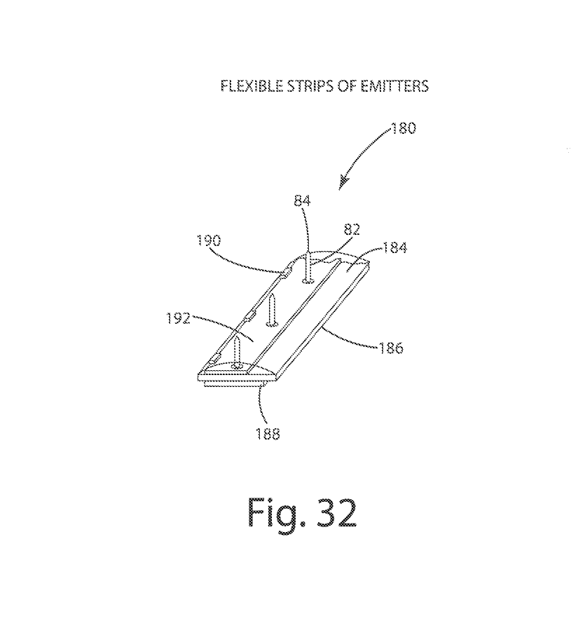

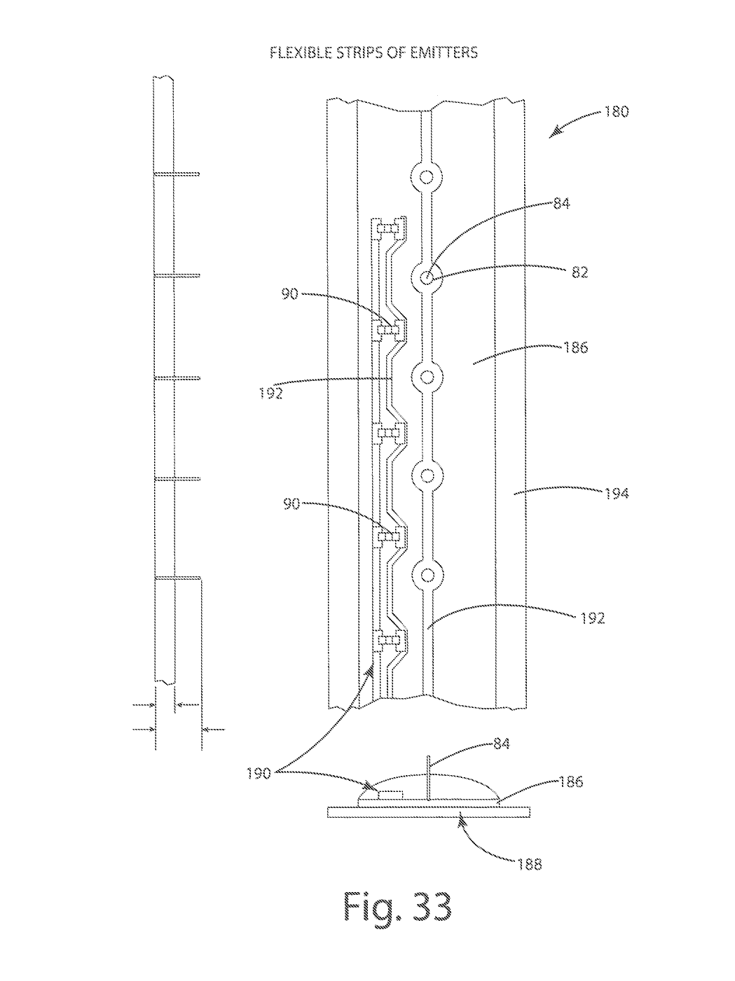

The points 84 are attached to a flexible circuit board 180 (FIG. 32). The inventors have discovered that it is possible to separately manufacture flexible circuit boards 180, flexible strips, or to modify commercially available LED light strips to provide both LED lighting and multiple points 84 (emitters) or alternatively to provide spaced points 84 only. These flexible circuit boards 180 or strips generally include a conductive strip 182 (e.g., copper) laminated with a dome 184 of urethane and affixed to a flexible polyamide dielectric material 186 such as Kapton having a pressure sensitive adhesive 188 disposed on one side to form the flexible circuit board 180. The flexible substrate or flexible polyamide dielectric material 186 could take on many other forms in other strips. The urethane forms a dome 184 on the top of the strip 180 to protect the circuit and helps support the emitters 80 or needles 84. LEDs 190 from the strip 180 may be removed and replaced with emitters 80 (e.g., stainless steel needles 84). The emitters 80 may be pressed into, soldered to, or epoxied to the strip 180 between LEDs 190 or in place of LEDs 190 and secured by various techniques, such as epoxy. The LEDs 190 may be controlled by the microprocessor 144 or by a separate controller. The LED strip in one ion generator 110 may be single color LEDs 190, but a separate ion generator 110 may use RGB multicolor LEDs 190 so that the perceived color from the strip can be adjusted to a myriad of colors.

A high voltage low current source can be connected to one end of the strip or flexible circuit board 180 with a suitable electrical connector. Ideally, the high voltage source is AC such that only a single row of connected discharge points is required. (DC would require two rows of discharge points, one positive and one negative) to create bipolar ionization. Alternately, a DC high voltage source such as the AC output 104 could be connected to the single row of discharge point to create positive or negative ions only, not both. In one ion generator 110, the reference ground 90 and emitter 80 (high voltage output) is connected to the LED light strip or separately manufactured strip with emitters 80 only. The high voltage AC output provides power to the emitters 80 attached to the strip as well as the LEDs 190.

The strips or flexible circuit boards 180 as described above may be mounted and used to sanitize, for example, a faucet, door handle, VFV/VRF heating, ventilation, and air conditioning systems, traditional heating, ventilation, and air conditioning systems. Furthermore, it could be used for under cabinet lighting with a counter sanitizer, refrigerator lighting and sanitizing, sanitizing and lighting a bread box, or toy box. The flexible nature of the strips allow them to be installed any area that needs sanitizing and/or lighting. The flexible discharge points 84 described in this invention are flexible and very small. The strips can be cut to any length with simple scissors for each installation in any application.

All flexible circuit boards 180, strips, or light strips include a flexible substrate or flexible dielectric polyamide material 186 to which conductive elements 192 are applied with spaced ion sources 82 being operationally coupled to the conductive element 192. The same conductive element 192 or additional conductive elements 192 may provide power to the LEDs 190 in addition to the ion sources 82. In addition, to form the strip, the flexible substrate may be applied to a flexible metallic material, such as an aluminum tape 194, which may act as the ground plane and in the embodiment an aluminum tape 194 may be easily adhered to various desired surfaces. While the Figures illustrate the ion sources 82 as protruding perpendicularly from such substrate, they may also be configured to extend parallel to the side.

In addition, the strip or flexible circuit board 180 may also include another conductive element 192 that acts as a ground electrode and is exposed to the atmosphere continuously or in selected portions. The strip will need to place the ion sources 82 at least ¼″ from such conductive ground electrode, which may cause the ion sources 82 to be located proximate to one edge and the ground electrode proximate to an opposing edge.

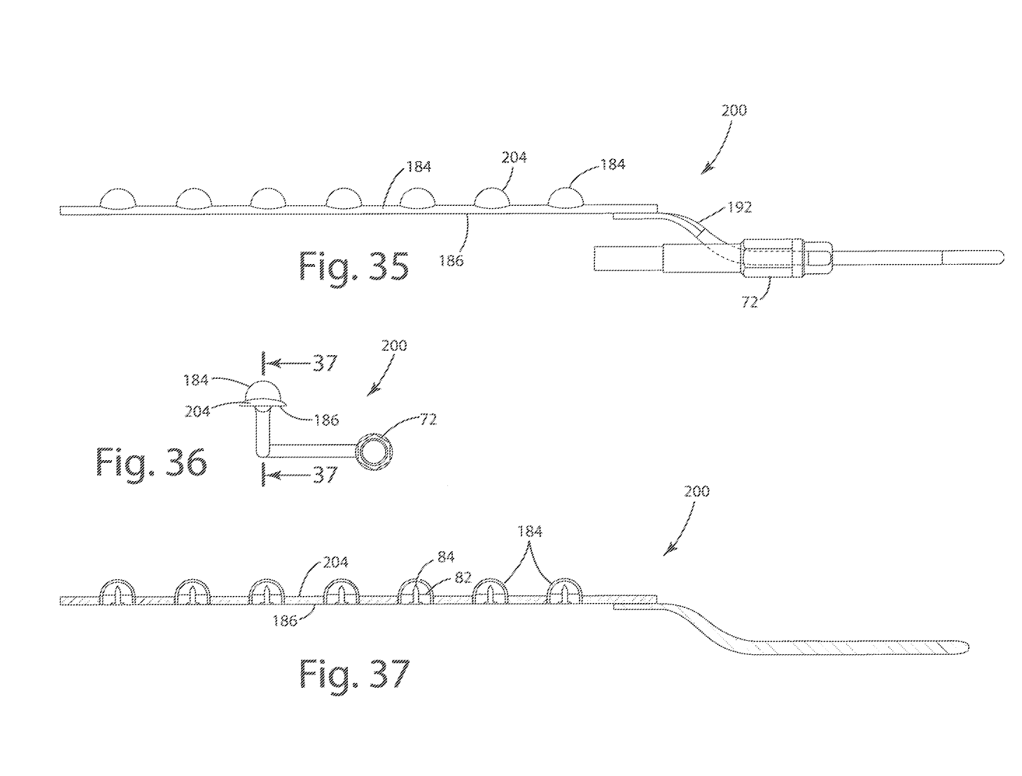

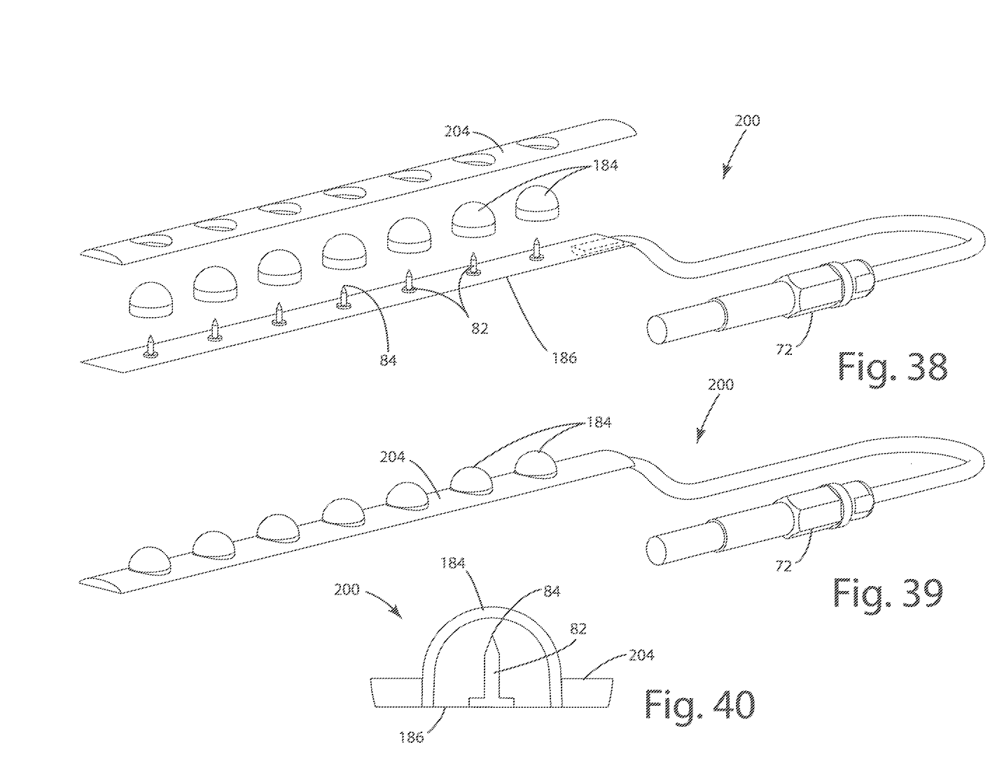

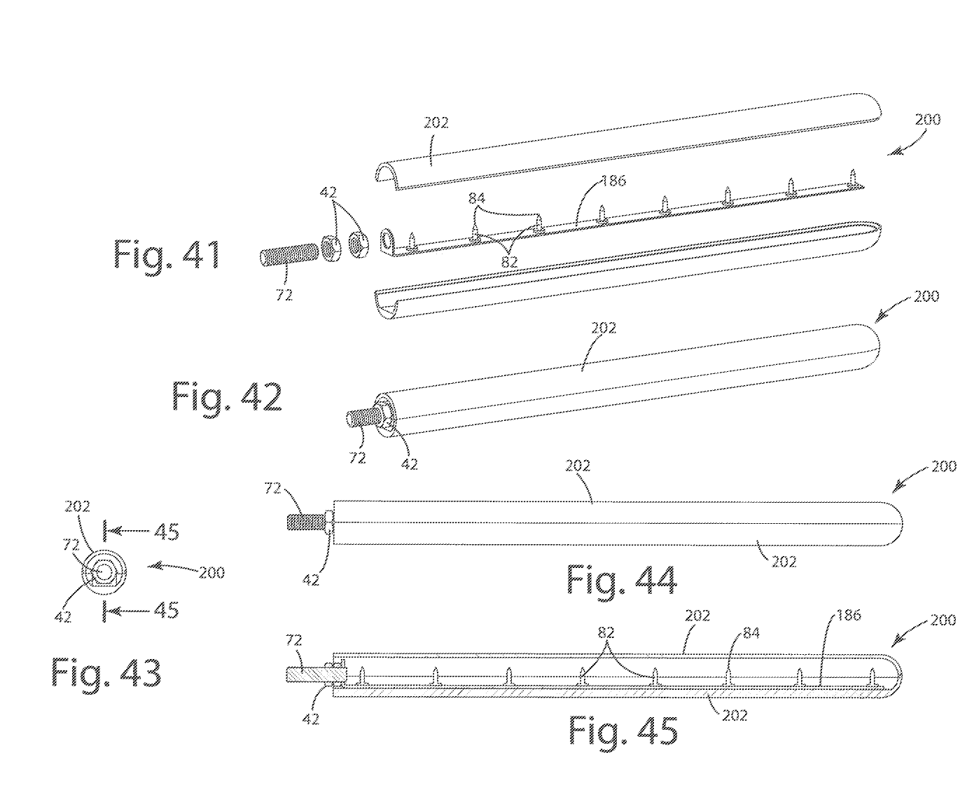

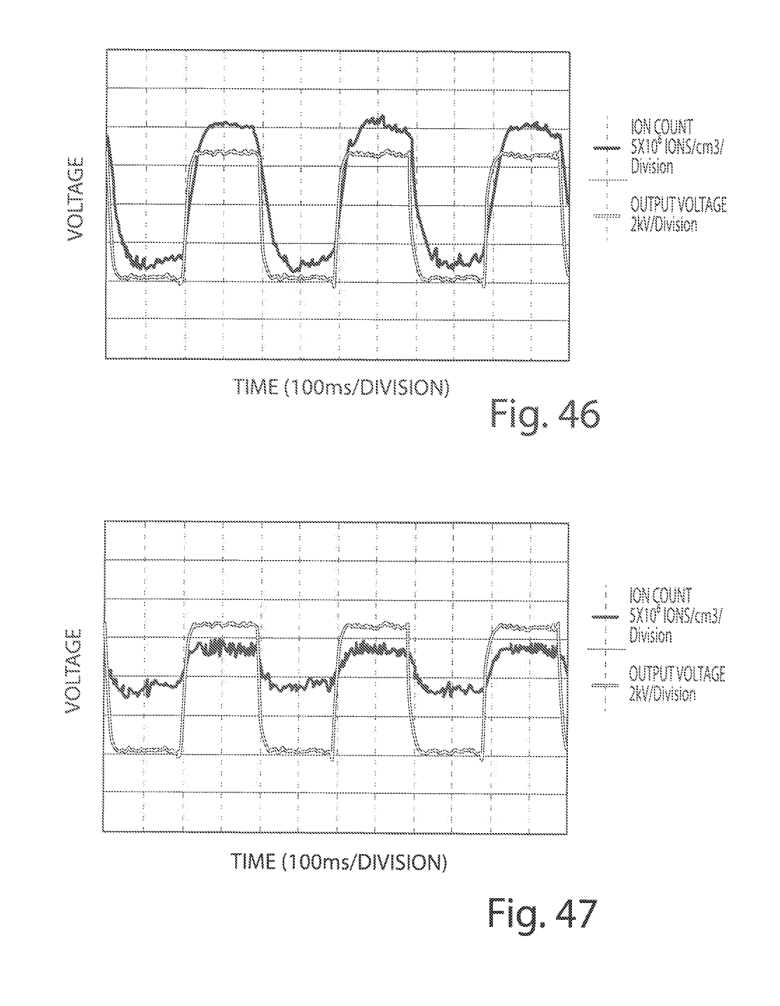

In addition, the present inventor has also surprisingly found that the ion sources may be covered by a cover or dome of thin wall of dielectric material such as plastic or glass, thereby preventing injury for contact with the points. Even though the ion generator runs at a voltage that is not harmful to touch, enclosing the ion sources and connective surfaces prevents any shorting of the ion electrode assembly in a wide range of environments, including those that experience moisture. Surprisingly the domes still allow a generous amount of ions to pass through and in reality the ion electrode assembly disclosed herein provides sufficient ions in a compact package that substantially outperforms prior art devices with exposed electrode. The domes in FIGS. 35-40 and covers in FIGS. 41-45 are formed from a nonconductive material. In the present invention, the nonconductive material of the domes and cover has surprisingly been found to provide more useable ions at the location where the ions are desired, such as at a surface, hands, in the air in an HVAC system, over a coil in an HVAC system and the like. Placed in an HVAC system, such as before the filter, could prevent the filter from growing pathogens and other undesirable organisms, likewise placed proximate to the coil in and HVAC system that regularly experiences moisture, the present invention also prevents the growth of harmful pathogens and organisms which then can be spread by the HVAC system. It is well known that cool damp areas in HVAC systems can have harmful effect on people breathing the air from such HVAC systems. In addition, for ductless mini-split systems, there is very limited ability to filter the air, and typically no ability to put in an efficient free flowing filter that is capable of filtering out most pathogens. As stated above, the filters need to be frequently cleaned or changed, such as every two weeks on most systems and most users do not follow this schedule, which allows pathogens and unwanted organisms to grow on the filters. In view of the filtration limits with ductless min-split systems, so it is even more important to keep the coil free from harmful organisms and pathogens.

As illustrated in FIGS. 35-40, the substrate is formed from a flexible material, such as an LED light strip, which allows reduction in shipping costs, as the material may be rolled, instead of being extended as required to ship rigid bars. The substrate generally includes a base material and a flexible conductive material applied thereto. The ion sources are in contact with the flexible conductive material. A substrate material this is nonconductive may be placed over the substrate and it and the domes may be injection molded. Of course, the ion electrode assembly could be formed without the substrate cover, the domes, or both. The ion electrode also includes a connector. Of course, modifications may be made to the ion electrode assembly as to size, shape, configuration.

FIGS. 41-45 represent an ion electrode assembly with a complete cover. While this will have greater length in shipping, the non-conductive cover provides the same function as the domes. The substrate may be formed from a flexible material, or may be formed from a ridged material, to which the ion sources are attached. As illustrated, the substrate is a metal plate with attached ion sources, such as through welding. The cover is placed over the substrate and a fastener, such as the illustrated connectors and fasteners may be used. This electrode is particularly useful in the duct work of an HVAC system, although the connector and fasteners need to be insulated from and not contact any metal ducts.

It should be noted that the covers and domes are only capable of being used with the ion generator of the present invention having AC source applied to the ion sources. The AC system with the ion electrode assembly creates a field that extends above and through the nonconductive domes and covers, creating the ions in the air around the domes and covers. One big benefit of this surprising revelation that sufficient ions are generated from ion sources that are covered and not exposed to the environment is that it eliminates the need to clean the ion sources. In prior embodiments, the ion sources would collect dust and debris that would substantially reduce their efficiency and require cleaning. Cleaning of the needles can cause injury from the sharp points, so the use of domes and covers that surprisingly do not reduce the efficiency of the ion fields is a major step forward in creating a maintenance free ion electrode assembly. While the ion sources may be covered with a solid dielectric material, it has been found that eventually the ion sources will burn a hole through the solid material, which opens them to the environment and opens them to moisture and dust which can reduce the efficiency. As such, the dome or cover has been developed, which creates and air gap, but surprisingly provides as good of an ion field outside of the cover or dome. The air gap prevents the burning of holes through the cover or domes, creating a seal over the ion sources that protect them from moisture and dust, therefore making them maintenance free and maintaining their effectiveness even in dirty environments. It has also been found that the dome or cover causes the ions to be distributed over a wider area, causing a more effective ion field, with all the benefits listed above and creating a shock resistant barrier between the environment and the ion sources.

Obviously, many modifications and variations of the present invention are possible in light of the above teachings and may be practiced otherwise than as specifically described while within the scope of the appended claims. These antecedent recitations should be interpreted to cover any combination in which the inventive novelty exercises its utility. The use of the word “said” in the apparatus claims refers to an antecedent that is a positive recitation meant to be included in the coverage of the claims whereas the word “the” precedes a word not meant to be included in the coverage of the claims. In addition, the reference numerals in the claims are merely for convenience and are not to be read in any way as limiting.

What is claimed is:

1. An ion sanitizer comprising:
a controller;
at least one ion electrode operationally coupled to said controller and wherein said ion electrode includes a plurality of ion sources spaced 6-51 mm apart; and
wherein at least a portion of said plurality of ion sources are covered with an electrical insulating material.

2. The ion sanitizer of claim 1 wherein said ion sanitizer defines a fixture cavity and wherein said plurality of ion sources each include a point directed toward said fixture cavity.

3. The ion sanitizer of claim 2 wherein said at least one ion electrode include a first ion electrode and a second ion electrode and wherein said controller provides a positive DC output to said first ion electrode, and a negative output to said second ion electrode and that said ion sources on said first and second electrode face each other and are each directed to said fixture cavity.

4. The ion sanitizer of claim 1 further including a ground electrode spaced at least 10 mm from the ion electrode, and wherein said ground electrode maintains a ground, while said ion electrode fluctuates between positive and negative charge at 1-100 Hz.

5. The ion sanitizer of claim 4 further including a housing and wherein said at least one ion electrode is recessed relative to the surface of the housing.

6. The ion sanitizer of claim 5 wherein said ion sources include a point and wherein said point is 0-4 mm recessed relative to said surface of said housing, and wherein the point does not protrude past said surface.

7. The ion sanitizer of claim 4 further including a housing wherein at least a portion of said housing forms said ground electrode.

8. The ion sanitizer of claim 1 further including a flexible substrate including at least one conductive element and wherein said ions sources are in electrical communication with said conductive element.

9. The ion sanitizer of claim 8 wherein said flexible substrate is coupled to a metallic base and wherein said metallic base is said ground electrode.

10. The ion sanitizer of claim 9 wherein said metallic base is a conductive metal tape capable of adhering said flexible substrate to a surface.

11. The ion sanitizer of claim 8 further including a plurality of LEDs coupled to said flexible substrate.

12. The ion sanitizer of claim 8 wherein said conductive element is covered with an electrical insulating material.

13. The ion sanitizer of claim 8 wherein said flexible substrate has a first longitudinal edge and an opposing second longitudinal edge and wherein said at least one conductive element includes a first conductive element in electrical communication with said ion sources and a second conductive element proximate to one of said first and second edges and wherein said second conductive element is a ground electrode spaced a minimum of 6 mm from said ion sources.

14. The ion sanitizer of claim 1 further including a housing having an outer extent, formed by at least one of a base and a cover and wherein said housing includes a recess on said outer extent configured to receive said at least one ion electrode.

15. The ion sanitizer of claim 14 wherein said ion electrode emits ions from 360 degrees of said outer extent.

16. An ion generator assembly comprising:
a microprocessor having a first PWM output and a second PWM output;
a first switching transistor connected to said first PWM output;
a second switching transistor connected to said second PWM output;
a first flyback transformer having a primary winding and a secondary winding and connected to said first switching transistor for generating a high-voltage peak;
a second flyback transformer having a primary winding and a secondary winding and connected to said second switching transistor for generating a high-voltage peak;
a first output section electrically connected to said secondary winding of said first flyback transformer for amplifying the high-voltage peak from said first flyback transformer;
a second output section electrically connected to said secondary winding of said second flyback transformer for amplifying the high-voltage peak from said second flyback transformer;
at least one emitter connected to said first output section and to said second output section and in communication with a reference plane for emitting positive and negative ions; and
said microprocessor configured to operate said first switching transistor and said second switching transistor at an operating frequency to generate a stable high voltage AC output at an output frequency and prevent unintended corona self-discharge and prevent the production of ozone during normal operation at said emitter.

17. An ion generator assembly as set forth in claim 16, wherein said microprocessor does not receive feedback to determine corona self-discharge at said emitter.

18. An ion generator assembly as set forth in claim 16, wherein said operating frequency is between 30 kHz and 400 kHz.

19. An ion generator assembly as set forth in claim 16, wherein said microprocessor is configured to intentionally create a corona discharge to vaporize dust and contaminants on said emitter.

20. An ion generator assembly as set forth in claim 16, wherein said reference plane is a fixture disposed adjacent said at least one emitter.

21. An ion generator assembly as set forth in claim 16, wherein said reference plane comprises ambient air surrounding said emitter.

22. An ion generator assembly as set forth in claim 16, further including at least one protection resistor disposed in series between said first output section and said emitter and between said second output section and said emitter for limiting electrical current to said emitter.

23. An ion generator assembly as set forth in claim 16, wherein said microprocessor is configured to output said second PWM output following said first PWM output after a delay time to create a dead zone to prevent the high-voltage peak of the first flyback transformer from cancelling out the high-voltage peak of the second flyback transformer.

24. An ion generator assembly as set forth in claim 23, wherein said delay time is between 2 milliseconds and 10 milliseconds.

25. An ion generator assembly as set forth in claim 16, wherein said first switching transistor and said first flyback transformer are configured to produce a positive half of said high voltage AC output and said second switching transistor and said second flyback transformer are configured to produce a negative half of said high voltage AC output.

26. An ion generator assembly as set forth in claim 16, wherein said output frequency of said high voltage AC output is between 10 Hz and 60 Hz.

27. An ion generator assembly as set forth in claim 16, wherein said first output section and said second output section each include a multiplier bridge comprising a plurality of capacitors and diodes arranged in a ladder configuration.

28. An ion generator assembly as set forth in claim 16, wherein said microprocessor is configured to intentionally create a temporary corona discharge and to vaporize dust and contaminants on said emitter.

29. An ion generator assembly as set forth in claim 16, further including a button coupled to said microprocessor for commanding one of an increase and a decrease in the high voltage AC output in incremental steps.

30. An ion generator assembly as set forth in claim 16, wherein said microprocessor is configured to vary the operation of said first switching transistor and said second switching transistor to adjust the voltage level of the high voltage AC output in response to at least one of a humidity level, a level of ionization, and air velocity.

31. An ion generator assembly as set forth in claim 16, further including a cover of dielectric material surrounding said emitter for preventing contact and injury with said emitter.

32. An ion generator assembly as set forth in claim 31, wherein said cover is a thin walled dome.

33. An ion generator assembly as set forth in claim 31, wherein said cover is spaced from said emitter and wherein said cover defines an air gap between said emitter and said cover.

34. An ion generator assembly as set forth in claim 16, wherein said emitter comprises a flexible circuit board and a plurality of points attached to said flexible circuit board.

35. An ion generator assembly as set forth in claim 34, wherein said flexible circuit board includes a conductive strip coupled to said plurality of points and a dome laminated to said conductive strip for insulating and protecting said conductive strip.

36. An ion generator assembly as set forth in claim 35, wherein said dome formed from urethane and said flexible circuit boards include a flexible polyamide dielectric material having a pressure sensitive adhesive disposed on one side.

37. An ion generator assembly as set forth in claim 35, wherein said flexible circuit board includes a plurality of LEDs attached to said flexible circuit board.

38. An ion generator assembly as set forth in claim 37, wherein said LEDs of said flexible circuit board are coupled to and controlled by said microprocessor.

39. An ion generator assembly as set forth in claim 37, wherein said LEDs are single color LEDs.

40. An ion generator assembly as set forth in claim 37, wherein said LEDs are multicolor LEDs.

41. An ion generator assembly as set forth in claim 35, further including a cover of dielectric material attached to said flexible circuit board for preventing contact and injury with said points.

42. An ion generator assembly as set forth in claim 41, wherein said cover is spaced from said points and wherein said cover defines an air gap between said points and said cover.

43. An ion generator assembly comprising:
a microprocessor configured to control a circuit to produce a high voltage AC output;
a flexible circuit board and a plurality of emitters attached to said flexible circuit board;
at least one emitter attached to said flexible circuit board and electrically connected to said circuit for receiving the high voltage AC output for emitting positive and negative ions;
said flexible circuit board includes a conductive strip coupled to said plurality of emitters and a dome of urethane laminated to said conductive strip for insulating and protecting said conductive strip;
said flexible circuit board formed of a flexible polyamide dielectric material having a pressure sensitive adhesive disposed on one side; and
a cover of dielectric material attached to said flexible circuit board for preventing contact and injury with said emitters.

44. An ion generator assembly as set forth in claim 43, further including a plurality of LEDs attached to said flexible circuit board.

45. A method of operating an ion generator assembly comprising:
outputting a first drive signal having a first duty cycle and operating frequency with a first PWM output of a microprocessor;
switching a first switching transistor with the first drive signal;
creating a high voltage peak with a first flyback transformer in response to the switching of the first switching transistor;
amplifying the high voltage peak from the first flyback transformer with a first multiplier bridge;
emitting positive ions from an emitter in response to the amplified high voltage peak from the first multiplier bridge;
delaying by a specified delay time;
outputting a second drive signal having a second duty cycle and operating frequency with a second PWM output of a microprocessor;
switching a second switching transistor with the second drive signal;
creating a high voltage peak with a second flyback transformer in response to the switching of the second switching transistor;
amplifying the high voltage peak from the second flyback transformer with a second multiplier bridge; and
emitting negative ions from an emitter in response to the amplified high voltage peak from the second multiplier bridge.

46. A method as set forth in claim 45, wherein the operating frequency is between 30 kHz and 400 kHz.

47. A method as set forth in claim 45, wherein the specified delay time is between 2 milliseconds and 10 milliseconds.

48. A method as set forth in claim 45, wherein the first duty cycle and the second duty cycle are unequal.

49. A method as set forth in claim 45, further including the step of controlling a plurality of LEDs with a microprocessor.

50. An emitter for an ion electrode comprising:
a base;
a plurality of ion sources extending from said base;
a cover extending over the majority of said ion sources; and
said cover being formed from an electrically insulating material.

51. An emitter as set forth in claim 50, wherein said cover is spaced from said ion sources and wherein said cover defines an air gap between said ion sources and said cover.

52. An emitter as set forth in claim 50, further including a flexible substrate including at least one conductive element and wherein said ion sources are in electrical communication with said conductive element.

53. An emitter for an ion electrode comprising:
a base;
a plurality of ion sources extending from said base;
a cover extending over the majority of said ion sources;
said cover being formed from an electrically conductive material; and
wherein said cover is spaced from said ion sources and wherein said cover defines an air gap between said ion sources and said cover.

54. An emitter as set forth in claim 53, wherein said cover is spaced from said ion sources and wherein said cover defines an air gap between said ion sources and said cover.

55. An emitter as set forth in claim 53, further including a flexible substrate including at least one conductive element and wherein said ion sources are in electrical communication with said conductive element.
